(19) United States
(12) Patent Application Publication
(10) Pub. No.: US 2023/0233647 A1
(43) Pub. Date: Jul. 27, 2023

(54) USE OF AN INHIBITOR OF ACTIN REMODELING MODULATOR FOR THE MANUFACTURE OF A MEDICAMENT FOR TREATMENT OF SLEEP DEPRIVATION-INDUCED MEMORY DEFICIT

(71) Applicant: TZU CHI UNIVERSITY, Hualien (TW)

(72) Inventors: Jasmin Sureka, Hualien (TW); Sarayut Phasuk, Hualien (TW); Prithiviraj Chokkalingam, Hualien (TW); Ingrid-Y Liu, Hualien (TW)

(21) Appl. No.: 17/697,263

(22) Filed: Mar. 17, 2022

(30) Foreign Application Priority Data
Jan. 27, 2022 (TW) 111103522

(51) Int. Cl. A61K 38/17; A61K 31/7105; A61K 45/06; A61K 9/00; A61P 25/28; A61P 25/20

(52) U.S. Cl. CPC A61K 38/1761; A61K 31/7105; A61K 45/06; A61K 9/0019; A61P 25/28; A61P 25/20

(57) ABSTRACT

The present disclosure concerns the use of an inhibitor of actin remodeling modulator thereof, for the preparation of a medicament for treating memory deterioration caused by sleep deprivation.

### REFERENCE TO RELATED APPLICATIONS

The present application is based on, and claims priority from, Taiwan application number 111103522 filed Jan. 7, 2022, the disclosure of which is hereby incorporated by reference herein in its entirety.

### TECHNICAL FIELD

The present disclosure relates to a use of an inhibitor of actin remodeling modulator thereof, for preparation of a medicament for treating memory deterioration caused by sleep deprivation.

### BACKGROUND OF RELATED ARTS

Sleep deprivation, also known as lack of sleep, is caused by physical fatigue due to voluntary insomnia, involuntary insomnia, or interruption of the sleep-wake cycle, making it difficult to maintain wakefulness. At present, sleep deprivation has been considered to be related to the occurrence of cognitive dysfunction such as reduced memory and learning functions, inattention, impaired judgment, etc., and causes possibility of accidents and human errors in daily life.

The Centers for Disease Control and Prevention (CDC) included sleep deprivation in the Behavioral Risk Factor Surveillance System (BRFSS) in 2009, according to the National Sleep Foundation. The recommended sleep time is that adults aged 18-64 need 7-9 hours of sleep per day, and those over 65 need 7-8 hours of sleep (Max Hirshkowitz 2015). However, the average sleep time is only 6.8 hours today. About half of the world's population suffers from sleep deprivation (Strine, T. W. 2005) (Chattu, V. K. 2018). In Taiwan, according to a survey conducted by the National Suicide Prevention Center (NSPC) in July 2020, about 25% of the population had experienced insomnia before the survey, which was a type of sleep disorder, and 4.2 million people use sleep aids for a long time.

Sleep has an important function of processing emotional memory and integrating storage for long-term memory (Cunningham, T. J. 2017). Studies have shown that complete sleep deprivation before memory acquisition and during memory consolidation impairs subsequent memory retrieval (retrieval) and reduces the correlation between the hippocampus and amygdala. Content of c-Fos, which is a neuronal activation indicator is related to memory retrieval and memory consolidation (Graves, L. A. 2003) (Montes-Rodriguez, C. J. 2019). Another study indicated that the use of CNS stimulants to induce sleep deprivation prior to fear memory reactivation impairs memory reconsolidation (Sharma, R. 2020).

Rapid eye movement (REM) sleep is necessary for learning and developmental processes, and REM sleep deprivation has been shown to affect brain regions important for memory formation and memory learning, including the hippocampus and the cortex of the brain (Prince, T. M. 2013). Studies have shown that sleep deprivation during rapid eye movement (REM) after spatial memory training or non-spatial memory training in rats can impair hippocampal-dependent memory, but not affect hippocampus-independent memory (Smith, C. 1997). Another study pointed out that reducing sleep time will reduce the content of postsynaptic density protein 95 (PSD-95), which is an important scaffold protein in synapses (Lopez, J. 2008). In addition, related to synaptic plasticity, the molecule brain-derived neurotrophic factor (BDNF), which acts upstream of PSD-95, is also susceptible to decline by REM sleep deprivation (Yoshii, A. 2014) (Schmitt, K. 2016). Sleep deprivation affects synaptic structure and affects the balance of actin regulators such as cofilin and profilin (Havekes, R. 2016) (Raven, F. 2019).

In conclusion, sleep deprivation may affect the structure and maintenance of synapses by altering the synaptic protein mechanism, thus causing the problem of memory degradation.

Although REM sleep deprivation is known to affect memory consolidation, the disruptive effects of REM sleep deprivation on memory retrieval and memory reconsolidation have not been fully elucidated. In addition, while there are currently studies trying to reveal the roles of profilin, cofilin and other actin-regulatory proteins in sleep and memory (Havekes, R. 2016) (Michaelsen-Preusse, K. 2016), no one has yet revealed gelsolin (gelsolin, GSN) in sleep and memory.

Actin, a protein with multiple functions, can form microfilament structures, which are structures necessary for cells to perform basic functions, including: movement, vesicle formaton, muscle contraction, signaling, and cell shape maintenance and so on (Dominguez, R. 2011). Actin is also present in neuronal cells, assisting in the formation of new synapses and inducing long-term potentiation (LTP) functions. Actin polymerization is also an important process of forming synaptic structure and promoting synaptic mobility to develop synaptic connections, and has been shown to be involved in the formation of long-term memory (Havekes, R. 2016), and both fear memory processing and synaptic transmission require actin in the hippocampus (Lamprecht, R. 2011).

The Fear Conditioning experiment is a habit of connecting animals to the fear caused by specific conditioned stimuli and unconditioned stimuli through association, so that animals are afraid of specific conditioned stimuli. Conditioning and fear responses were linked to study and assess the effects of memory learning (Sanders, M. J. 2003). Memories that link animals to specific conditioned stimuli and fear responses through training are stored in the cortex, and when the animal is re-exposed to conditioned stimuli, the consolidated memory is retrieved through a hippocampus-dependent pathway that evokes fear responses (Izquierdo, I., C. 2016). Even over a period of days, these memories can be revived by retrieval and reconsolidation of remote fear memories, possibly with gradual extinction during memory extinction (Myers, K. M. 2007). Therefore, the function of memory in memory retrieval, memory reconsolidation and remote fear memory retrieval can be studied through fear conditioning experiments.

The formation of fear memory is related to the functions of memory retrieval, reconsolidation and remote memory retrieval through synapse-like kinase signaling molecules. Kinases such as extracellular regulated protein kinase (ERK) and phosphoinositol triphosphate kinase (PI3K), and neurotropic molecules such as brain-derived neurotrophic factor (BDNF) are necessary to fear memory formation and retrieval (Liu, I. Y. 2004) (Chen, X. 2005) (Antoine, B. 2014). Brain-derived neurotrophic factor precursor (pro-BDNF) can be activated by enzymes to form mature brain-derived neurotrophic factor (mature BDNF, m-BDNF), and by interacting with tropomyosin receptor kinase B receptor (TrKB receptor) activates structural proteins such as the mechanistic target of rapamycin (mTOR) through the phosphoinositol triphosphate kinase/protein kinase B pathway (PI3K/AKT pathway) (Hempstead B L. 2015); and it can also be mediated by Ca²⁺/calmodulin-dependent protein kinase II (CaMKII)/cAMP responsive element-binding protein (CREB) signaling regulates self-gene expression (Cunha, C. 2010). Molecules such as BDNF, protein kinase B (AKT) and CAMKII are known to play an important role in the integration and processing of received information into long-term memory by the sensory system (Itoh, N. 2016).

Among them, after AKT is phosphorylated, it can further phosphorylate downstream factors and promote synaptic plasticity; and after CaMKII is phosphorylated, it can phosphorylate the upstream molecules of synapsin 1 (synapsin I, SYN 1), and CAMKII contains 4 subtypes: α type, β type, δ type and γ type, and α type and β type are the subtypes with brain specificity. SYN 1 is a pre-synaptic protein marker that is involved in trafficking of synaptic vesicle and release of neurotransmitter after phosphorylation (Wang, Z.-W. 2008) (Zalcman G. 2018). Therefore, the detection of phosphorylated AKT (p-AKT), the phosphorylated CAMKII (p-CAMKII) and the phosphorylated SYN 1 (p-SYN 1) in tissues can be used to determine the performance of synaptic function. The higher the contents of the phosphorylated CAMKII and the phosphorylated SYN 1, the more active the synaptic function.

At present, there are many factors for the molecular mechanism of sleep deprivation-induced memory deficit, including apoptosis, neuroinflammation, neurogenesis, oxidative stress, epigenetic modification and cytoskeleton remodeling (Nelson, J. C. 2013) (Mirescu, C. 2006) (Wessel M A van Leeuwen 2009) (Lahtinen, A. 2019) (Wong, L. W. 2019) (Vaccaro, A. 2020).

Gelsolin (GSN) is a protein of 82 kilodaltons (kDa), and its function is a regulator of actin modulating protein. It exists in the human body in two forms, namely cytosolic (cytosol) and plasma (plasma), and both types of GSN are derived from the same gene (alternative splicing) (Wang, W. 2019). GSN caps filamentous actin (F-actin), which depolymerizes filamentous actin into monomeric globular actin (G-actin) (Angliker, N. 2013). In the brain, GSN is found in neurons and oligodendrocytes (Michaelsen-Preusse, K. 2016) (Kamali, A. 2016), and has the ability to reduce inflammation in the brain and inhibit glia Function in glioblastoma (Kruijssen, D. L. H. 2019) (Fitzgerald, P. J. 2015). However, the effect of GSN on memory and synaptic plasticity has not yet been elucidated.

### REFERENCES

1. Hirshkowitz, M., et al., National Sleep Foundation's sleep time duration recommendations: methodology and results summary. Sleep Health, 2015. 1(1): p. 40-43.

2. Strine, T. W. and D. P. Chapman, Associations of frequent sleep insufficiency with health-related quality of life and health behaviors. Sleep Med, 2005. 6(1): p. 23-7.

3. Chattu, V. K., et al., The Global Problem of Insufficient Sleep and Its Serious Public Health Implications. Healthcare (Basel), 2018. 7(1).

4. Cunningham, T. J. and J. D. Payne, Emotional Memory Consolidation During Sleep, in Cognitive Neuroscience of Memory Consolidation, N. Axmacher and B. Rasch, Editors. 2017, Springer International Publishing: Cham. P. 133-159.

5. Graves, L. A., et al., Sleep deprivation selectively impairs memory consolidation for contextual fear conditioning. Learn Mem, 2003. 10(3): p. 168-76.

6. Montes-Rodriguez, C. J., P. E. Rueda-Orozco, and O. Prospéro-Garcia, Total sleep deprivation impairs fear memory retrieval by decreasing the basolateral amygdala activity. Brain Research, 2019. 1719: p. 17-23.

7. Sharma, R., P. Sahota, and M. M. Thakkar, Sleep Loss Immediately After Fear Memory Reactivation Attenuates Fear Memory Reconsolidation. Neuroscience, 2020. 428: p. 70-75.

8. Prince, T. M. and T. Abel, The impact of sleep loss on hippocampal function. Learn Mem, 2013. 20(10): p. 558-69.

9. Smith, C. and G. M. Rose, Posttraining paradoxical sleep in rats is increased after spatial learning in the Morris water maze. Behav Neurosci, 1997. 111(6): p. 1197-204.

10. Lopez, J., et al., Rapid eye movement sleep deprivation decreases long-term potentiation stability and affects some glutamatergic signaling proteins during hippocampal development. Neuroscience, 2008. 153(1): p. 44-53.

11. Yoshii, A. and M. Constantine-Paton, Postsynaptic localization of PSD-95 is regulated by all three pathways downstream of TrkB signaling. Front Synaptic Neurosci, 2014. 6: p. 6.

12. Schmitt, K., E. Holsboer-Trachsler, and A. Eckert, BDNF in sleep, insomnia, and sleep deprivation. Ann Med, 2016. 48(1-2): p. 42-51.

13. Havekes, R., et al., Sleep deprivation causes memory deficits by negatively impacting neuronal connectivity in hippocampal area CA1. eLife, 2016. 5: p. e13424.

14. Raven, F., et al., A brief period of sleep deprivation causes spine loss in the dentate gyrus of mice. Neurobiology of Learning and Memory, 2019. 160: p. 83-90.

15. Michaelsen-Preusse, K., et al., Neuronal profilins in health and disease: Relevance for spine plasticity and Fragile X syndrome. 2016. 113(12): p. 3365-3370.

16. Dominguez, R. and K. C. Holmes, Actin structure and function. Annu Rev Biophys, 2011. 40: p. 169-86.

17. Lamprecht, R., The roles of the actin cytoskeleton in fear memory formation. Front Behav Neurosci, 2011. 5: p. 39.

18. Sanders, M. J., B. J. Wiltgen, and M. S. Fanselow, The place of the hippocampus in fear conditioning. Eur J Pharmacol, 2003. 463(1-3): p. 217-23.

19. Izquierdo, I., C. R. G. Furini, and J. C. Myskiw, Fear Memory. 2016. 96(2): p. 695-750.

20. Myers, K. M. and M. Davis, Mechanisms of fear extinction. Molecular Psychiatry, 2007. 12(2): p. 120-150.

21. Liu, I. Y., et al., Brain-derived neurotrophic factor plays a critical role in contextual fear conditioning. J Neurosci, 2004. 24(36): p. 7958-63.

22. Chen, X., et al., PI3 kinase signaling is required for retrieval and extinction of contextual memory. Nature Neuroscience, 2005. 8(7): p. 925-931.

23. Antoine, B., L. Serge, and C. Jocelyne, Comparative dynamics of MAPK/ERK signalling components and immediate early genes in the hippocampus and amygdala following contextual fear conditioning and retrieval. Brain Structure and Function, 2014. 219(1): p. 415-430.

24. Hempstead B L. Brain-Derived Neurotrophic Factor: Three Ligands, Many Actions. Trans Am Clin Climatol Assoc. 2015;126:9-19. PMID: 26330656; PMCID: PMC4530710.

25. Cunha, C., R. Brambilla, and K. L. Thomas, A simple role for BDNF in learning and memory? Front Mol Neurosci, 2010. 3: p. 1.

26. Itoh, N., et al., Molecular mechanism linking BDNF/TrkB signaling with the NMDA receptor in memory: the role of Girdin in the CNS. Reviews in the Neurosciences, 2016. 27(5): p. 481-490.

27. Wang, Z.-W., Regulation of synaptic transmission by presynaptic CaMKII and BK channels. Molecular neurobiology, 2008. 38(2): p. 153-166.

28. Zalcman G, Federman N, Romano A. CaMKII Isoforms in Learning and Memory: Localization and Function. Front Mol Neurosci. 2018 Dec 4;11:445. Doi: 10.3389/fnmol.2018.00445. PMID: 30564099; PMCID: PMC6288437.

29. Nelson, J. C., A. K. H. Stavoe, and D. A. Colón-Ramos, The actin cytoskeleton in presynaptic assembly. Cell adhesion & migration, 2013. 7(4): p. 379-387.

30. Mirescu, C., et al., Sleep deprivation inhibits adult neurogenesis in the hippocampus by elevating glucocorticoids. Proceedings of the National Academy of Sciences, 2006. 103(50): p. 19170.

31. van Leeuwen, W. M., et al., Sleep restriction increases the risk of developing cardiovascular diseases by augmenting proinflammatory responses through IL-17 and CRP. PloS One, 2009. 4(2): p. e4589.

32. Lahtinen, A., et al., A distinctive DNA methylation pattern in insufficient sleep. Scientific Reports, 2019. 9(1): p. 1193.

33. Wong, L. W., et al., The p75 Neurotrophin Receptor Is an Essential Mediator of Impairments in Hippocampal-Dependent Associative Plasticity and Memory Induced by Sleep Deprivation. J Neurosci, 2019. 39(28): p. 5452-5465.

34. Vaccaro, A., et al., Sleep Loss Can Cause Death through Accumulation of Reactive Oxygen Species in the Gut. Cell, 2020. 181(6): p. 1307-1328.e15.

35. Wang, W., E. Halasz, and E. Townes-Anderson, Actin Dynamics, Regulated by RhoA-LIMKCofilin Signaling, Mediates Rod Photoreceptor Axonal Retraction After Retinal Injury. Investigative Ophthalmology & Visual Science, 2019. 60(6): p. 2274-2285.

36. Angliker, N. and M. A. Rüegg, In vivo evidence for mTORC2-mediated actin cytoskeleton rearrangement in neurons. Bioarchitecture, 2013. 3(4): p. 113-8.

37. Kamali, A., et al., Methodological Aspects of REM Sleep-Deprivation and Stereological Protocols in the Brain-Stem Respiratory Nuclei 2016.

38. Kruijssen, D. L. H. and C. J. Wierenga, Single Synapse LTP: A Matter of Context? 2019. 13(496).

39. Fitzgerald, P. J., et al., Durable fear memories require PSD-95. Mol Psychiatry, 2015. 20(7): p. 901-12.

### SUMMARY OF THE INVENTION

In view of the fact that sleep deprivation is a prevalent problem in the world, and there is no drug to treat the problem of memory degradation caused by sleep deprivation, an object of the present disclosure is to solve the memory degradation caused by sleep deprivation.

According to the purpose of the present disclosure, the purpose of providing an inhibitor of actin recombination regulator is to prepare a medicament for the treatment of memory deterioration caused by sleep deprivation.

Wherein, the actin recombination regulator is gelsolin.

Wherein, the inhibitor of actin recombination regulator includes short hairpin RNA (shRNA), microRNA (miRNA), small interfering RNA (siRNA), antibody, antagonist or combination thereof.

Wherein, the mode of administration of the inhibitor of actin recombination regulator is selected from the group consisting of: intracerebroventricular administration, intracerebral administration, intrathecal administration, arterial administration, intradermal administration, intramuscular administration Administration, intragastric administration, intraperitoneal administration, intravenous administration, oral administration, subcutaneous administration, topical administration, systemic administration.

Wherein, the inhibitor of further actin recombination regulator can be used in combination with hypnotic drug.

Wherein, the hypnotic drug is selected from the group consisting of benzodiazepines, non-benzodiazepines, barbiturates, and melatonin receptor agonists.

In conclusion, the present disclosure can improve memory degradation caused by sleep deprivation.

### BRIEF DESCRIPTION OF THE DRAWINGS

FIG. 1 shows the experimental procedure of contextual fear conditioning (CFC).

FIG. 2 is a quantitative histogram of the percentage of freezing reaction time in the experimental mice at different time points, including: CFC, Ret-1, Ret-2, and Ret-3.

FIG. 3 is a graph of results of fEPSP measurement of the long-term potentiation experiment.

FIG. 4 is a quantitative histogram merged with the dot chart of the graph of results of fEPSP measurement of the long-term potentiation experiment.

FIG. 5 is a graph showing the relationship between the amplitude change of fEPSP (unit of amplitude change: millivolt (mV)) and stimulation intensity (unit of stimulation intensity: microampere (μA)).

FIG. 6 is a graph of paired pulse ratio (PPF ratio) obtained by performing pair pulse facilitation (PPF) at different stimulation intervals.

FIG. 7 is a schematic diagram of the experimental procedure for determining the effect of sleep deprivation on brain presynaptic transmission impairment at the molecular level.

FIG. 8A shows the development map of the content of phosphorylated SYN 1 (p-SYN 1), the total content of SYN 1, and glyceraldehyde-3-phosphate dehydrogenase (GAPDH) in the hippocampus of the experimental mice analyzed by Western blot analysis.

FIG. 8B shows the development map of the content of phosphorylated α isoform CAMKII (p-CAMKIIα) and phosphorylated β isoform CAMKII (p-CAMKIIβ) and the content of GAPDH in the hippocampus of the experimental mice analyzed by Western blot analysis.

FIG. 8C shows a quantitative histogram merged with the dot chart of FIG. 8A showing the content of p-SYN 1 in the hippocampus of the experimental mice analyzed by Western blot analysis, after correction by the content of GAPDH.

FIG. 8D shows the quantitative histogram merged with the dot plot of FIG. 8A showing the total content of SYN 1 in the hippocampus of the experimental mice analyzed by Western blot analysis analysis, after correction by the content of GAPDH.

FIG. 8E shows the quantitative histogram merged with the dot plot of FIG. 8B showing the content of p-CAMKIIα and the content of p-CAMKIIβ in the hippocampus of the experimental mice analyzed by Western blot analysis, after correction by the content of GAPDH.

FIG. 9A is a graph showing the fluorescence staining of p-SYN 1 in the CA1 of the hippocampus of the experimental mice by immunofluorescence staining analysis.

FIG. 9B is a quantitative histogram merged with the dot plot of FIG. 9A showing the fluorescence staining of p-SYN 1 in the CA1 of the hippocampus of the experimental mice by immunofluorescence staining analysis.

FIG. 10A is a graph showing the fluorescence staining of p-SYN 1 in the CA2 of the hippocampus of the experimental mice by immunofluorescence staining analysis.

FIG. 10B is a quantitative histogram merged with the dot plot of FIG. 10A showing the fluorescence staining of p-SYN 1 in the CA2 of the hippocampus of the experimental mice by immunofluorescence staining analysis.

FIG. 11A is a graph showing the fluorescence staining of p-SYN 1 in the CA3 of the hippocampus of the experimental mice by immunofluorescence staining analysis.

FIG. 11B is a quantitative histogram merged with the dot plot of FIG. 11A showing the fluorescence staining of p-SYN 1 in the CA3 of the hippocampus of the experimental mice by immunofluorescence staining analysis.

FIG. 12A is a graph showing the fluorescence staining of p-SYN 1 in the dentate gyrus (DG) of the hippocampus of the experimental mice by immunofluorescence staining analysis.

FIG. 12B is a quantitative histogram merged with the dot plot of FIG. 12A showing the fluorescence staining of p-SYN 1 in the dentate gyms (DG) of the hippocampus of the experimental mice by immunofluorescence staining analysis.

FIG. 13A is a graph showing the fluorescence staining of p-SYN 1 in the cortex of the hippocampus of the experimental mice by immunofluorescence staining analysis.

FIG. 13B is a quantitative histogram merged with the dot plot of FIG. 13A showing the fluorescence staining of p-SYN 1 in the cortex of the hippocampus of the experimental mice by immunofluorescence staining analysis.

FIG. 14A is a graph showing the fluorescence staining of p-SYN 1 in the amygdala of the hippocampus of the experimental mice by immunofluorescence staining analysis.

FIG. 14B is a quantitative histogram merged with the dot plot of FIG. 14A showing the fluorescence staining of p-SYN 1 in the amygdala of the hippocampus of the experimental mice by immunofluorescence staining analysis.

FIG. 15 is a schematic diagram of the experimental procedure for determining whether the content of gelsolin (GSN) and related proteins changes before memory retrieval.

FIG. 16A shows the development map of the content of GSN, phosphorylated AKT (p-AKT), and GAPDH in the hippocampus of the experimental mice analyzed by Western blot analysis before memory retrieval performing on 2 hours after training.

FIG. 16B shows the development map of the contents of mature BDNF (m-BDNF) and postsynaptic density protein 95 (PSD-95) and GAPDH in the hippocampus of the experimental mice analyzed by Western blot analysis before memory retrieval performing on 2 hours after training.

FIG. 16C is a quantitative histogram merged with the dot plot of FIG. 16A showing the content of GSN in the hippocampus of the experimental mice analyzed by Western blot analysis before memory retrieval performing on 2 hours after training, after correction by the contents of GAPDH.

FIG. 16D is a quantitative histogram merged with the dot plot of FIG. 16A showing the content of p-AKT in the hippocampus of the experimental mice analyzed by Western blot analysis before memory retrieval performing on 2 hours after training, after correction by the contents of GAPDH.

FIG. 16E is a quantitative histogram merged with the dot plot after correction by the contents of GAPDH of FIG. 16B showing the content of m-BDNF in the hippocampus of the experimental mice analyzed by Western blot analysis before memory retrieval performing on 2 hours after training.

FIG. 16F is a quantitative histogram merged with the dot plot of FIG. 16B showing the content of PSD-95 in the hippocampus of the experimental mice analyzed by Western blot analysis before memory retrieval performing on 2 hours after training, after correction by the contents of GAPDH.

FIG. 17 is a schematic diagram of the experimental procedure for determining whether the content of GSN and related proteins changes after remote fear memory retrieval.

FIG. 18A shows the development map of the contents of GSN and GAPDH in the hippocampus of the experimental mice analyzed by Western blot analysis after remote fear memory retrieval.

FIG. 18B shows the development map of the p-AKT and GAPDH in the hippocampus of the experimental mice analyzed by Western blot analysis after remote fear memory retrieval.

FIG. 18C shows the development map of the contents of m-BDNF, PSD-95, and GAPDH in the hippocampus of the experimental mice analyzed by Western blot analysis after remote fear memory retrieval.

FIG. 18D is a quantitative histogram merged with the dot plot after correction by the contents of GAPDH of FIG. 18A showing the contents of GSN in the hippocampus of the experimental mice analyzed by Western blot analysis after remote fear memory retrieval.

FIG. 18E is a quantitative histogram merged with the dot plot of FIG. 18B showing the contents of p-AKT in the hippocampus of the experimental mice analyzed by Western blot analysis after remote fear memory retrieval, after correction by the contents of GAPDH.

FIG. 18F is a quantitative histogram merged with the dot plot of FIG. 18C showing the contents of m-BDNF in the hippocampus of the experimental mice analyzed by Western blot analysis after remote fear memory retrieval, after correction by the contents of GAPDH.

FIG. 18G is a quantitative histogram merged with the dot plot of FIG. 18C showing the contents of PSD-95 in the hippocampus of the experimental mice analyzed by Western blot analysis after remote fear memory retrieval, after correction by the contents of GAPDH.

FIG. 19A shows the fluorescence staining of GSN in whole brain sections of the experimental mice in the NSD group after the remote fear memory retrieval test.

FIG. 19B shows the fluorescence staining of GSN in whole brain sections of the experimental mice in SD group after the remote fear memory retrieval test.

FIG. 20A is a graph showing the fluorescence staining of GSN of CA1 in hippocampal slices of the experimental mice analyzed by immunofluorescence staining after the remote fear memory retrieval test.

FIG. 20B is a quantitative histogram merged with the dot plot of FIG. 20A showing the fluorescence staining of GSN of CA1 in hippocampal slices of the experimental mice analyzed by immunofluorescence staining after the remote fear memory retrieval test.

FIG. 21A is a graph showing the fluorescence staining of GSN of CA2 in hippocampal slices of the experimental mice analyzed by immunofluorescence staining after the remote fear memory retrieval test.

FIG. 21B is a quantitative histogram merged with the dot plot of FIG. 21A showing the fluorescence staining of GSN of CA2 in hippocampal slices of the experimental mice analyzed by immunofluorescence staining after the remote fear memory retrieval test.

FIG. 22A is a graph showing the fluorescence staining of GSN of CA3 in hippocampal slices of the experimental mice analyzed by immunofluorescence staining after the remote fear memory retrieval test.

FIG. 22B is a quantitative histogram merged with the dot plot of FIG. 22A showing the fluorescence staining of GSN of CA3 in hippocampal slices of the experimental mice analyzed by immunofluorescence staining after the remote fear memory retrieval test.

FIG. 23A is a graph showing the fluorescence staining of GSN of the superior granular layer, the inferior granular layer, the global granular layer, and the hilus of the dentate gyrus (DG) in hippocampal slices of the experimental mice analyzed by immunofluorescence staining after the remote fear memory retrieval test.

FIG. 23B is a quantitative histogram merged with the dot plot of FIG. 23A showing the fluorescence staining of GSN of the superior granular layer, the inferior granular layer, the global granular layer, and the hilus of the dentate gyms (DG) in hippocampal slices of the experimental mice analyzed by immunofluorescence staining after the remote fear memory retrieval test.

FIG. 23C is a quantitative histogram merged with the dot plot of FIG. 23A showing the fluorescence staining of GSN of the inferior granular layer of DG in hippocampal slices of the experimental mice analyzed by immunofluorescence staining after the remote fear memory retrieval test.

FIG. 23D is a quantitative histogram merged with the dot plot of FIG. 23A showing the fluorescence staining of GSN of the whole granular layer of DG in hippocampal slices of the experimental mice analyzed by immunofluorescence staining after the remote fear memory retrieval test.

FIG. 23E is a quantitative histogram merged with the dot plot of FIG. 23A showing the fluorescence staining of GSN of the hilus in hippocampal slices of the experimental mice analyzed by immunofluorescence staining after the remote fear memory retrieval test.

FIG. 24A is a graph showing the fluorescence staining of GSN of the external granular layer, the external pyramidal layer, and the internal granular layer in brain cortical slices of the experimental mice analyzed by immunofluorescence staining after the remote fear memory retrieval test.

FIG. 24B is a quantitative histogram merged with the dot plot of FIG. 24A showing the fluorescence staining of GSN of the external granular layer in brain cortical slices of the experimental mice analyzed by immunofluorescence staining after the remote fear memory retrieval test.

FIG. 24C is a quantitative histogram merged with the dot plot of FIG. 24A showing the fluorescence staining of GSN of the external pyramidal layer in brain cortical slices of the experimental mice analyzed by immunofluorescence staining after the remote fear memory retrieval test.

FIG. 24D is a quantitative histogram merged with the dot plot of FIG. 24A showing the fluorescence staining of GSN of the internal granular layer in brain cortical slices of the experimental mice analyzed by immunofluorescence staining after the remote fear memory retrieval test.

FIG. 25A shows the fluorescence staining of GSN in the amygdala of the experimental mice analyzed by immunofluorescence staining after the remote fear memory retrieval test.

FIG. 25B is a quantitative histogram merged with the dot plot of FIG. 25A showing the fluorescence staining of GSN in amygdala of the experimental mice analyzed by immunofluorescence staining after the remote fear memory retrieval test.

FIG. 26A shows a graph of immunohistochemical staining of the filamentous actin (F-actin) in CA1, CA3 and DG of the hippocampus of the experimental mice analyzed by immunohistochemical staining after the remote fear memory retrieval test.

FIG. 26B is a quantitative histogram merged with the dot plot of FIG. 26A showing immunohistochemical staining of the F-actin in CA1 of the hippocampus of the experimental mice analyzed by immunohistochemical staining after the remote fear memory retrieval test.

FIG. 26C is a quantitative histogram merged with the dot plot of FIG. 26A showing immunohistochemical staining of the F-actin in CA3 of the hippocampus of the experimental mice analyzed by immunohistochemical staining after the remote fear memory retrieval test.

FIG. 26D is a quantitative histogram merged with the dot plot of FIG. 26A showing immunohistochemical staining of the F-actin in DG of the hippocampus of the experimental mice analyzed by immunohistochemical staining after the remote fear memory retrieval test.

FIG. 27A shows the development map of the content of GSN and GAPDH in the hippocampus and the amygdala of the experimental mice of the SD group injected with GSN siRNA analyzed by Western blot analysis.

FIG. 27B is a quantitative histogram merged with the dot plot of FIG. 27A showing the content of GSN in the hippocampus of the experimental mice of the SD group injected with GSN siRNA analyzed by Western blot analysis, after correction by the content of GAPDH.

FIG. 28A shows the development map of the content of GSN in the hippocampus and the amygdala of the experimental mice of the SD group injected with GSN siRNA analyzed by Western blot analysis.

FIG. 28B is a quantitative histogram merged with the dot plot of FIG. 28A showing the content of GSN in the hippocampus of the experimental mice of the SD group injected with GSN siRNA analyzed by Western blot analysis, after correction by the content of GAPDH.

FIG. 29 is a schematic diagram of the experimental procedure of the contextual fear conditioning experiment after GSN siRNA was injected to the the experimental mice of the SD group.

FIG. 30 is a quantitative histogram merged with the dot plot of percentage of freezing reaction time obtained by the contextual fear conditioning experiment after the experimental mice in the SD group were injected with GSN siRNA at different time points, including: CFC, Ret-1, Ret-2, and Ret-3.

### DETAILED DESCRIPTION OF THE PREFERRED EMBODIMENTS

As described herein, “a” or “an” means one or at least one.

As described herein, “about”, “nearly” or “approximately” generally means within the range of 20%, preferably 10%, and most preferably 5%. Numerical values herein are approximations, and the meaning of “about”, “nearly” or “approximately” may be implied where not explicitly defined.

The small interfering RNA (siRNA) described in this embodiment is a double-stranded RNA molecule with a length of 20 to 25 bases, which can be passed through RNA interference (RNAi) pathway to suppress the expression of genes complementary to the siRNA sequence.

Except for the above-mentioned definitions, the technical or scientific terms used in this specification are all the general definitions related to the present disclosure understood by those with ordinary knowledge in the field.

In view of the fact that sleep deprivation is a prevalent problem in the world, and there is no drug to treat the problem of memory degradation caused by sleep deprivation, an object of the present disclosure is to solve the memory degradation caused by sleep deprivation. In order to achieve the purpose of the present disclosure, the present disclosure provides the use of an inhibitor of actin recombination regulator, which is used to prepare a medicament for treating memory deterioration caused by sleep deprivation.

In a preferred embodiment of the present disclosure, the actin recombination regulator is gelsolin.

In a preferred embodiment of the present disclosure, the inhibitor of actin recombination regulator includes shRNA, miRNA, siRNA, antibody, antagonist or combination thereof.

In a preferred embodiment of the present disclosure, the mode of administration of the inhibitor of actin recombination regulator is selected from the group consisting of: intracerebroventricular administration, intracerebral administration, intrathecal administration, arterial administration, intradermal administration, intramuscular administration, intragastric administration, intraperitoneal administration, intravenous administration, oral administration, subcutaneous administration, topical administration, systemic administration.

In a preferred embodiment of the present disclosure, the inhibitor of further actin recombination regulator can be used in combination with hypnotic drug.

In a preferred embodiment of the present disclosure, the hypnotic drug is selected from the group consisting of benzodiazepines, non-benzodiazepines, barbiturates, and melatonin receptor agonists.

In order to understand the content of the present disclosure more clearly, specific embodiments of the present disclosure are described in detail below with reference to the accompanying drawings.

An example is provided below that provides an exemplary protocol for the use of inhibitors of the modulator of actin recombination for the treatment of sleep deprivation-induced memory degradation.

The data results presented in the following examples are graphed with the mean as the center and the standard deviation, and the Student's t-test is used to compare whether the experimental results between the two groups are statistically significant, wherein, statistical significance is defined as p<0.05, which is represented by “*” in the drawings of the following embodiments, and when p≥0.05, it means that there is no significant difference, which is represented by “ns”.

The experimental animals used in this example were C57BL/B6 wild-type male mice provided by the National Laboratory Animal Center in Taiwan, hereinafter referred to as the experimental mice. The experimental mice were cared for in the experimental animal center of Tzu Chi University (Taiwan). The experimental mice had free access to food and drinking water, and were in a 7:7 light-dark cycle (L/D cycle). The zeitgeber time (ZT) time was defined that starts at 7:00 a.m. which is defined as ZTO, other times such as 8:00 a.m. as ZT1, 9:00 a.m. as ZT2, and so on. All treatment of experimental mice was reviewed and approved by the Institutional Animal Care and Use Committee of Tzu Chi University.

In this example, a rapid eye movement (REM) sleep deprivation experimental mouse model was first established, and a contextual fear conditioning (CFC) experiment was used to determine the effect of the established REM sleep deprivation on the formation of fear memory. Then, long-term potentiation (LTP) experiments were performed to assess synaptic plasticity. Next, it was determined whether the contents of gelsolin (GSN) and related proteins changed before memory retrieval. Then, the location of gelsolin distribution in the brain after the remote fear memory retrieval test was determined. Next, it was determined whether the content of GSN and related proteins changes before remote fear memory retrieval. Then, the location of gelsolin distribution in the brain after the remote fear memory retrieval test was determined. Next, it was determined whether sleep deprivation caused actin depolymerization after the remote fear memory retrieval test. Then, it was determined whether reducing the content of GSN could improve the problem of memory deterioration caused by sleep deprivation.

1. Establishment of REM Sleep Deprivation Experimental Mouse Model:

In the establishment of the REM sleep deprivation experimental mouse model, the experimental mice were divided into two groups after contextual fear training, namely the sleep-deprived (SD) group and the non-sleep-deprived (NSD) group. The SD group was placed in multiple-platform chambers for sleep deprivation treatment from 7 a.m. (ZT0) to 11 a.m. (ZT4). Among them, the multi-platform room had at least one circular platform with a diameter of 2.5 cm and a height more than 2.5 cm. First, after placing the experimental mice in the SD group into the multi-platform, water at a depth of 2.5 cm was injected into the multi-platform chamber. Based on the characteristics of the experimental mice's aversion to water and the loss of muscle tension when they entered the REM phase, they were not able to maintain standing on the platform, so that a mouse model of REM sleep deprivation was established (Kamali, A. 2016).

Contextual Fear Conditioning (CFC) Experiment:

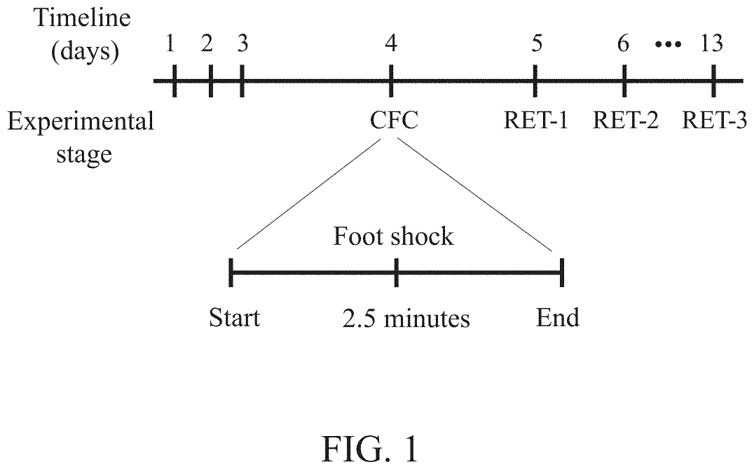

In the contextual fear conditioning experiment, the experimental mice were first placed in a conditioning chamber for 15 minutes/day for 3 days to allow the experimental mice to adapt to the conditioning chamber environment. On the 4th day, the experimental mice were subjected to the contextual fear conditioning experiment, and the experimental mice were allowed to form the memory of the aversive events. Among them, the aversive event was that when the experimental mice were placed in the conditioned chamber for 2.5 minutes, a single 0.3 milliampere (mA) foot shock was given to the experimental mice for 2 seconds. After the 3rd minute, the experimental mice were removed from the conditioned room, and the percentage of the freezing reaction time of the experimental mice was observed during the period, and the percentage of the freezing reaction time of the experimental mice to aversive events was obtained. The experimental stage is hereinafter referred to as CFC. On the 5th day, the experimental mice were placed in the conditioning chamber for a 5-minute fear contextual test, without foot shocks during the period. The percentage of the experimental mice's freezing reaction time was observed, and the experimental mice's reaction to aversion events was obtained, hereinafter referred to as the experimental stage Ret-1. On the 6th day, the experimental mice were placed in the conditioning chamber for a 5-minute contextual test, without foot shocks during the period. The percentages of the experimental mice's freezing reaction time were observed, and the experimental mice's reactions of reconsolidation to aversion events were obtained, hereinafter referred to as the experimental stage Ret-2. On the 13th day, the experimental mice were placed in the conditioning chamber for a 5-minute contextual test, without foot shocks during the period. The percentages of the experimental mice's freezing reaction time were observed, and the experimental mice's reactions of remote fear memory retrieval to aversion events were obtained, hereinafter referred to as the experimental stage Ret-3. The experimental procedure is shown in FIG. 1.

2. Confirm the Effect of REM Sleep Deprivation on the Formation of Fear Memory:

After establishing a sleep deprivation mouse model, in order to determine the effect of REM sleep deprivation on the formation of fear memory, contextual fear conditioning experiments were performed on the experimental mice in the NSD group and SD group, and it was determined that in different experimental stages, including: CFC, Ret-1, Ret-2, and Ret-3, the percentage of time that the experimental mice produced a freezing reaction to evaluate the memory function of the experimental mice.

The experimental values are expressed as the percentage of freezing reaction time in the contextual test, and the percentage of freezing reaction time is calculated: the percentage of freezing reaction time (%)=(total freezing time/total contextual test time)×100.

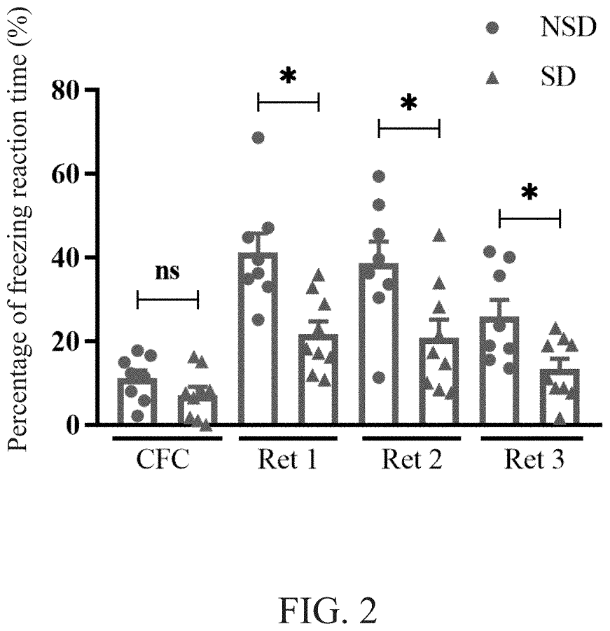

FIG. 2 shows that in the CFC experimental stage (NSD group: n=8; SD group: n=9), there was no significant difference (p>0.05) between the NSD group and the SD group. Among them, in the Ret-1 experimental stage, the percentage of freezing reaction time in SD group was significantly lower than that in NSD group (p=0.003). Among them, in the Ret-2 experimental stage, the percentage of freezing reaction time in SD group was significantly lower than that in NSD group (p=0.01). Among them, in the Ret-3 experimental stage, the percentage of freezing reaction time in SD group was significantly lower than that in NSD group (p=0.01). The results showed that the experimental mice in the SD group were impaired in the ability to retrieve, reconsolidate, and retrieve the remote fear memory.

3. Evaluation of Synaptic Plasticity by Long-Term Potentiation (LTP) Experiments:

After determining that REM sleep deprivation impaired the ability to retrieve fear memory, reconsolidate fear memory, and retrieve the remote fear memory. Then, Next, synaptic plasticity was assessed by long-term potentiation experiments.

The long-term potentiation experiment was used to evaluate synaptic plasticity, that is, through continuous and rapid action potential transmission to the terminal of the presynaptic neuron, so that the neurotransmitter was released from the terminal of the presynaptic neuron to trigger post-synaptic neuronal depolarization responses. Then, the long-term enhancement of the signalling strength between the presynaptic neuron and the postsynaptic neuron (Kruijssen, D. L. H. 2019) was used to assess synaptic plasticity, as well as memory and learning functions.

After completing the recording of the fear conditioning experiment, the experimental mice in each group were subjected to head decapitation and the brains were removed. After removing the brain, the brains were immediately placed in ice-cold artificial cerebrospinal fluid (ACSF) to cool for 3 to 5 minutes. Next, the brains of the experimental mice were cut into slices with a thickness of about 350 micrometers (μm) using a vibrating microtome (Micro slicer DTK-1000, Dosaka EM Co. Ltd., Kyoto, Japan). The slices are stored in ACSF with continuous bubbling at 2-3 milliliters per minute (mL/min) for 2 hours at 28° C.

In the long-term potentiation experiment, a recording electrode was placed in the CA1 region of the hippocampus to record field excitatory postsynaptic potential (fEPSP). Unipolar stainless-steel microelectrodes (Frederick Haer Company, Bowdoinham, Me., USA) were used as stimulation electrodes. The stimulus intensity for each slice was adjusted at 3-10 volts (V) to evoke 30-40% of the fEPSP maximal response intensity. First, the experiment was evoked every 20 seconds for the first 10 minutes or 20 minutes, with the same stimulation intensity and frequency; the average value of fEPSP measured during the period was used as the control group, which is hereinafter referred to as the baseline. High-frequency stimulation (HFS) was performed after the baseline recordings were completed. Among them, HFS was stimulated at 100 hertz (Hz) for 60 seconds, followed by stimulation every 20 seconds to induce fEPSP for 60 minutes. The results were divided by the decreasing slope of the measured fEPSP by the decreasing slope of the baseline, and expressed as a percentage, which is abbreviated as “percentage of the decreasing slope of fEPSP” in the figure. The recording signal was amplified by an amplifier (Axon Multiclamp 700B amplifier), the filter signal threshold was set to 1 kilohertz (kHz), and a signal digitization software was used through a signal conversion interface (CED Micropower 1401 MKII interface, Cambridge Electronic Design, Cambridge, UK). The downward slope of fEPSP was recorded. If the fEPSP was maintained at a level higher than the baseline after HFS, it means that the synaptic signal transmission is good. If the fEPSP after HFS gradually approached the baseline level over time, it means the synaptic signal transmission function damaged.

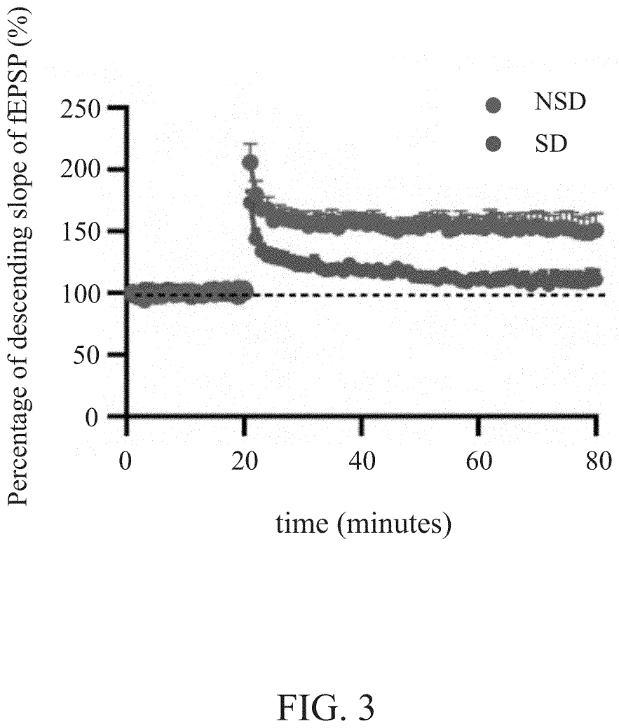

FIG. 3 shows HFS performed on the experimental mice in NSD and SD groups at 100 hertz (Hz) (NSD: n=8 slices/4 experimental mice; SD: n=9 slices/4 experimental mice). The decline slope of fEPSP at 80 minutes after the end of HFS in the experimental mice in the NSD group was maintained at about 1.5 times that of the baseline. Gradually decline to a level similar to the descending slope of the baseline. The results demonstrated that the synaptic transmission ability of the experimental mice in the SD group was impaired.

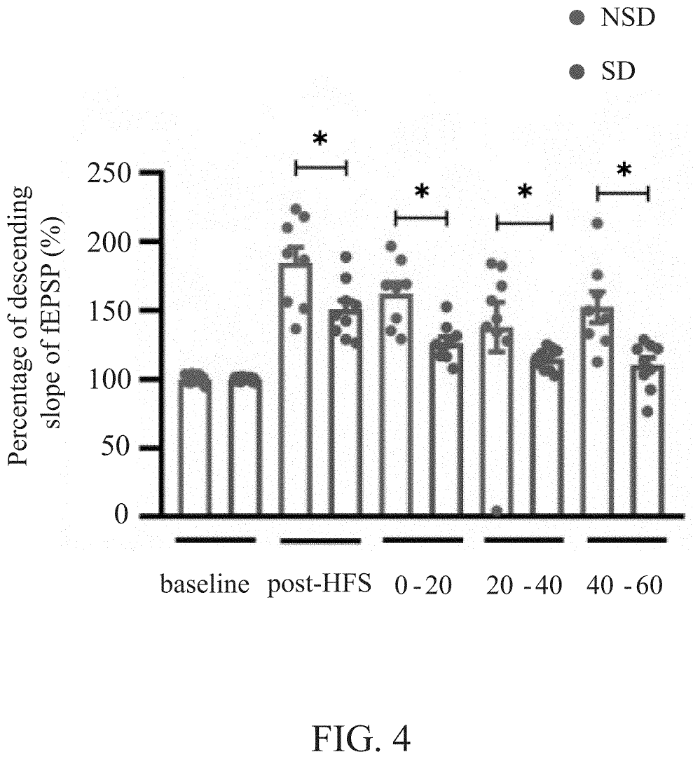

FIG. 4 shows each time point or each time interval (baseline: baseline; post-HFS: post-high frequency stimulation; 0-20: 0-20 minutes; 20-40: 20-40 minutes; 40-60: 40-60 minutes), the descending slope of fEPSP is expressed as a percentage relative to the descending slope of the baseline, and is expressed as the quantitative histogram merged with the dot plot. Among them, after HFS, 0-20, 20-40, and 40-60 groups, the descending slopes of fEPSP of the experimental mice in SD group were significantly lower than those in NSD group. The results demonstrated that the synaptic transmission ability of the experimental mice in the SD group was impaired.

4. Determination of the Effect of REM Sleep Deprivation on the Synaptic Plasticity of Remote Fear Memory Retrieval Process:

After confirming that REM sleep deprivation impairs synaptic transmission, the effect of REM sleep deprivation on synaptic plasticity during remote fear memory retrieval was determined.

In order to determine the effect of REM sleep deprivation on the synaptic plasticity of the remote fear memory retrieval process, extra-cellular recording was performed on the experimental mice of each group after the test of remote fear memory retrieval. The system assessed the basal neurotransmission ability and presynaptic function by measuring the amplitude changes of fEPSP in the hippocampus with different stimulation intensities, as well as pair pulse facilitation (PPF) experiments.

Among them, the basal nerve conduction capacity was used to evaluate the basal transmission efficiency of the experimental mouse synapse through the range of different stimulation intensities, and it was plotted as the amplitude change of fEPSP (amplitude change unit: mV) and stimulation intensity (stimulation intensity unit: μA) relationship diagram.

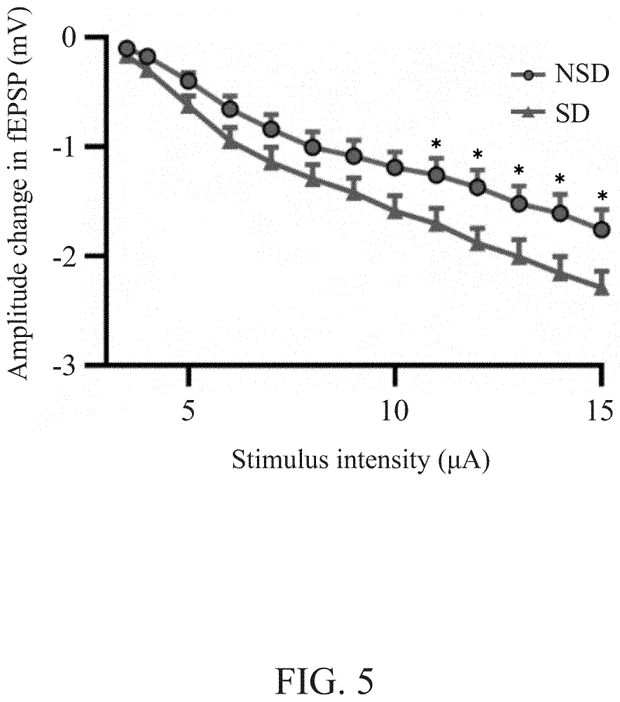

FIG. 5 shows the relationship between the amplitude change (amplitude change unit: mV) of fEPSP and stimulus intensity (stimulus intensity unit: μA) after the remote fear memory retrieval test (NSD: n=10 slices/4 experimental mice; SD: n=4 slices/3 experimental mice). The amplitude changes of fEPSP of the experimental mice in SD group at each stimulation intensity were larger than those of the experimental mice in NSD group, and the amplitude changes of fEPSP was greater than 10 microamps (μA). Under the stimulation intensity greater than 10 microamperes (μA), the amplitude change of fEPSP of the experimental mice in SD group was significantly smaller than that of the experimental mice in NSD group at each signal acquisition time point. The results demonstrated that the experimental mice in the SD group could not maintain the basal nerve conduction ability.

The pair pulse facilitation (PPF) experiment, performed after the remote fear memory retrieval recording, was used to confirm short-term synaptic plasticity and to determine postsynaptic reaction. The recording method of the PPF experiment is the same as the aforementioned long-term potentiation experiment, except that in the PPF experiment, the hippocampus of the experimental mice in the NSD group and the SD group were subjected to different stimulation intervals (15, 30, 50, 100, 150, 200, and 250 milliseconds (ms)), and the stimulation intensity was increased to 3.5-15 mA to evoke 40-60% of the fEPSP maximal response intensity. The trace figure of paired pulse ratio (PPF ratio) was recorded at each different stimulation interval (NSD: n=8 slices/4 experimental mice; SD: n=10 slices/4 experimental mice).

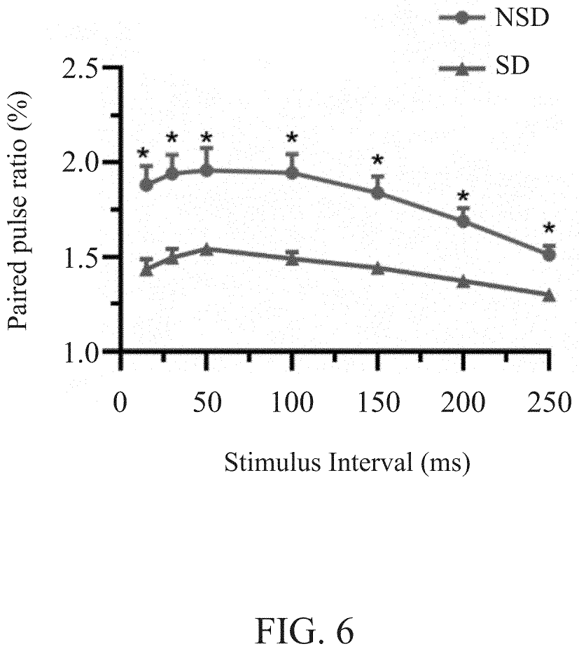

FIG. 6 shows a graph of paired pulse ratio (PPF ratio) for the experimental mice in the NSD group and SD group at each stimulation interval (NSD: n=8 slices/4 experimental mice; SD: n=10 slices/4 experimental mice) mouse). It was found that the PPF ratio in SD group was lower than that in NSD group in each stimulation interval from 15 to 250 ms. The results demonstrated impaired short-term synaptic plasticity in the experimental mice in the SD group.

5. Determination of the Molecular-Level Effects of Sleep Deprivation Causing Impaired Presynaptic Transmission in the Brain:

After determining that the synaptic plasticity of REM sleep deprivation in the process of remote fear memory retrieval resulted in the failure to maintain basic nerve conduction capacity and the impairment of short-term synaptic plasticity, we further determined that sleep deprivation causes the molecular-level impact of sleep deprivation in impaired presynaptic transmission in the brain.

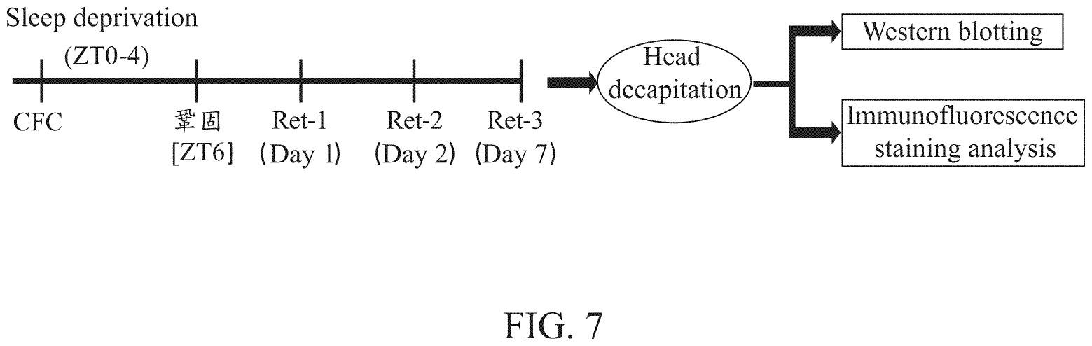

To determine the protein content of phosphorylated SYN 1 and phosphorylated CAMKII, the experimental mice were subjected to brain sections after remote fear memory retrieval, confirmed using Western blot analysis and immunofluorescence staining analysis. FIG. 7 is a schematic diagram of the experimental procedure.

Protein Extraction and Perfusion:

The brains of the experimental mice were taken out after head decapitation, and the hippocampus was taken out and immersed in 500 μL of radioimmunoprecipitation buffer (RIPA buffer). The hippocampus was then centrifuged at 13,000 rpm for 15 minutes at 4° C. to isolate the protein, and the isolated protein was stored at −20° C. The brain was extracted by myocardial perfusion method using 0.9% normal saline and 4% paraformaldehyde fix solution (PFA). The extracted brains were stored in 4% PFA for 2 days, then transferred to sucrose solution and stored at 4° C.

Western Blot Analysis:

In Western blot analysis, first, protein samples were 10-fold diluted for quantification by Bradford protein assay to remove 30 micrograms (μg) of sample into microcentrifuge tubes. Then, electrophoresis was performed with 10% or 12% sodium dodecyl sulfate polyacrylamide gel electrophoresis (SDS-PAGE). After electrophoresis at 80V for 20 minutes, electrophoresis at 140V for 60 minutes was used to separate proteins of different molecular weights by gel electrophoresis. Next, the proteins were transferred from the gel to polyvinylidene difluoride (PVDF) using a transfer system for 2 hours at 4° C. Next, PVDF was blocked with 5% milk or 1% bovine serum albumin (BSA) for 1 hour. Next, primary antibodies were added according to the type of protein to be observed, and were diluted with phosphate-buffered saline with tween 20 (PBST) according to the appropriate dilution ratio of different antibodies. The protein targets and dilution ratios of primary antibodies are as follows: GSN (1:500) (Cell signaling Technology, Inc., USA), phosphorylated AKT (p-AKT) (1:1000) (Cell signaling Technology, Inc., USA), PSD-95 (1:1000) (Thermo Fisher Scientific Inc., USA), glyceraldehyde-3-phosphate dehydrogenase (GAPDH) (1:5000, GeneTex, Inc., USA), BDNF (1:1000, Cell signaling Technology, Inc., USA), SYN 1 (1:2000, Cell signaling

Technology, Inc., USA), and phosphorylated SYN 1 (p-SYN 1) (1:2000, Cell signaling Technology, Inc., USA), reacted with PVDF at 4° C. for 18 hours. Next, the PVDF was rinsed 3 times for 10 minutes with tris-buffered saline with tween 20 (TBST). Next, the secondary antibody was diluted 1:10000 with 0.1% milk-TBST and the washed PVDF was reacted for 10 minutes at room temperature. Finally, after immersing the PVDF in an electrochemiluminescence (ECL) developing solution for 5 minutes in the dark and reacting in the dark, the development results on the PVDF are captured by a cold light image capture and analysis system (WS-High Sensitivity program). Data quantification of development results was analyzed with Image J software.

Immunofluorescence Analysis:

In the immunofluorescence analysis, first, the brain slices were immersed in 0.1% PFA for preservation, then rinsed with cold phosphate buffered saline (Phosphate-Buffered Saline, PBS) for 3 minutes. The permeation buffer was composed of 1% Triton X-100 (Triton X-100) and 2% TBST. Then, the sections were immersed in blocking agent and reacted at room temperature for 60 minutes. The blocking agent was 1% normal goat serum (NGS) and PBS containing 0.3% Triton X-100. Next, the anti-GSN primary antibody was diluted 50-fold, and the anti-p-SYN 1 primary antibody was diluted 100-fold using an antibody dilution buffer. The dilution buffer was composed of 1% NGS and PBS containing 0.25% Triton X-100. Next, the blocking agent was removed and the diluted primary antibody was added before reacting overnight in the refrigerator and then using washing buffer to wash 3 times for 5 minutes each time. The washing buffer was PBS containing 0.25% Triton X-100. Next, the secondary antibody (1:200) was diluted with the antibody dilution buffer. Next, after removing washing buffer the secondary antibody was added to react in the dark at room temperature for 1 to 2 hours so that the secondary antibody binds to the primary antibody, and then using washing buffer to wash 3 times for 5 minutes each time. Next, a 5 mg/mL solution of 4′,6-diamidino-2-phenylindole (DAPI) was prepared using PBS. Then, after removing the washing and rinsing solution, DAPI solution was added to react in the dark at room temperature for 1 hour. Slice imaging was observed using a confocal microscope. Data were analyzed with image processing software (Image J software) and graphed with graphing software (GraphPad Prism 8). Image cropping and contrast adjustment were performed using an image processing software (Adobe photoshop), in which DAPI was used for nuclear staining, and the color was blue fluorescence in the picture. The secondary antibody has a green fluorescent group or a red fluorescent group, so in the following immunofluorescence staining analysis scheme, it is displayed as green fluorescence or red fluorescence. The “percentage of fluorescent stained area” described in the following figures refers to the percentage of green fluorescence distribution area in the photographed area, or the percentage of the red fluorescence distribution area in the photographed area.

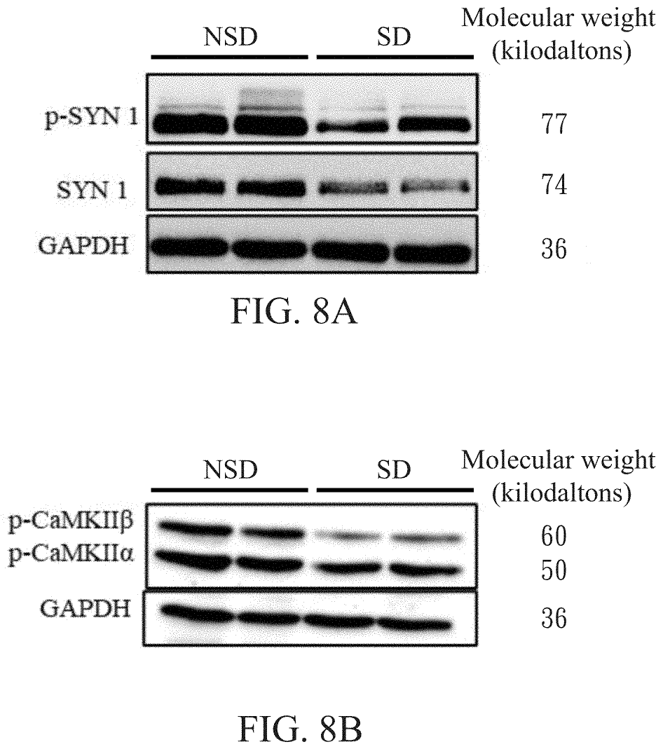

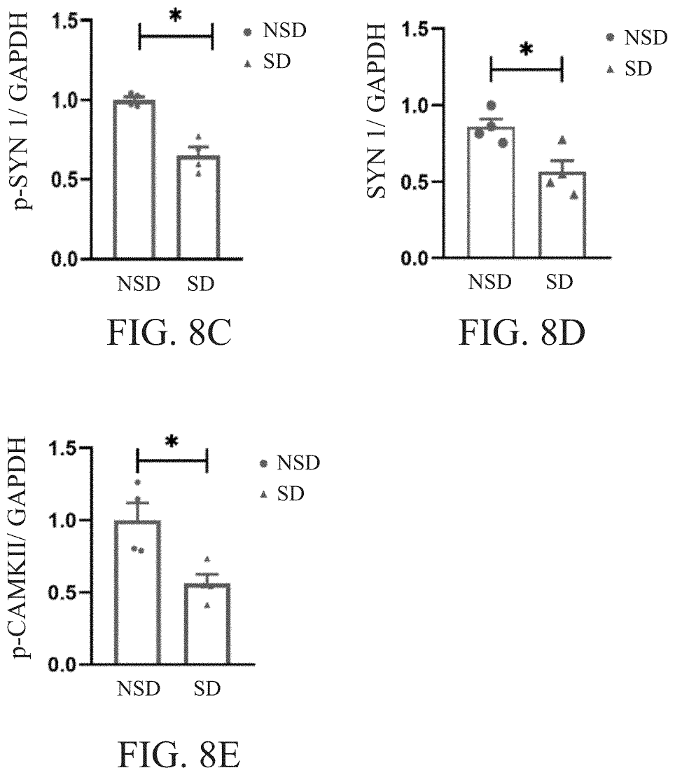

FIG. 8A, FIG. 8B, FIG. 8C, FIG. 8D, and FIG. 8E show that in the Western blot analysis, There was no significant difference in the content of GAPDH in the experimental mice in the SD group (n=3 slices/3 experimental mice) as an internal control group. The phosphorylated SYN 1 (p-SYN 1) content (p=0.0007) after correction by GAPDH content (p=0.0007) (FIG. 8C), the overall SYN 1 content (p=0.003) (FIG. 8D) and the content of p-CAMKII (p=0.018) (FIG. 8E) were significantly lower than those of the experimental mice in the NSD group. The results demonstrate impaired presynaptic function in the experimental mice in the SD group. The “content of p-CAMKII” is an abbreviation for the content of phosphorylated α isoform CAMKII (p-CAMKIIα) combined with the content of phosphorylated β isoform CAMKII (p-CAMKIIβ).

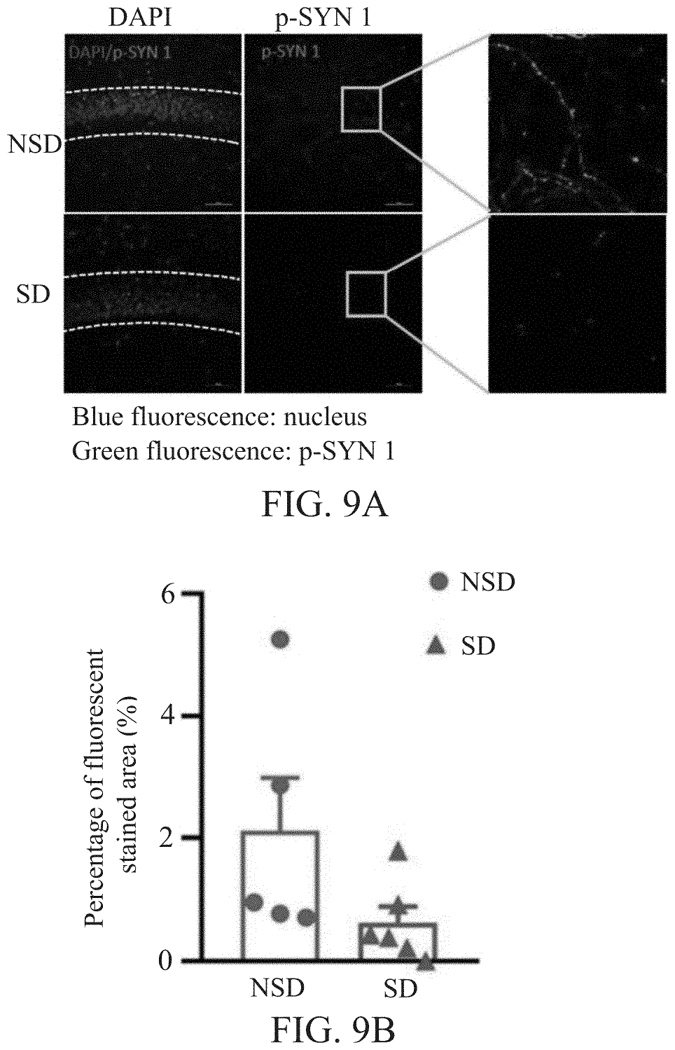

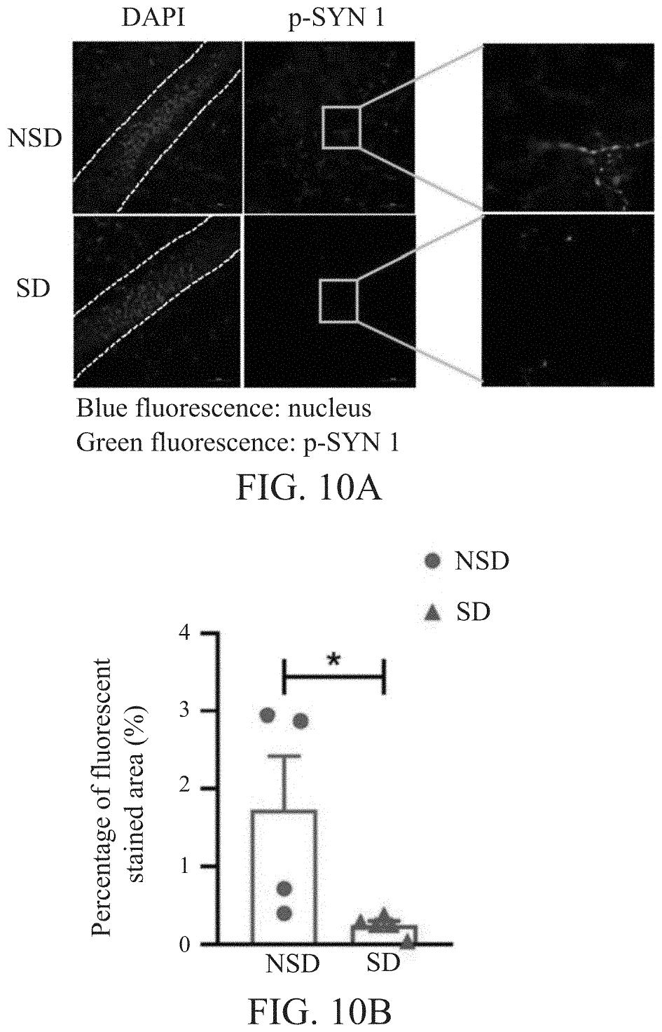

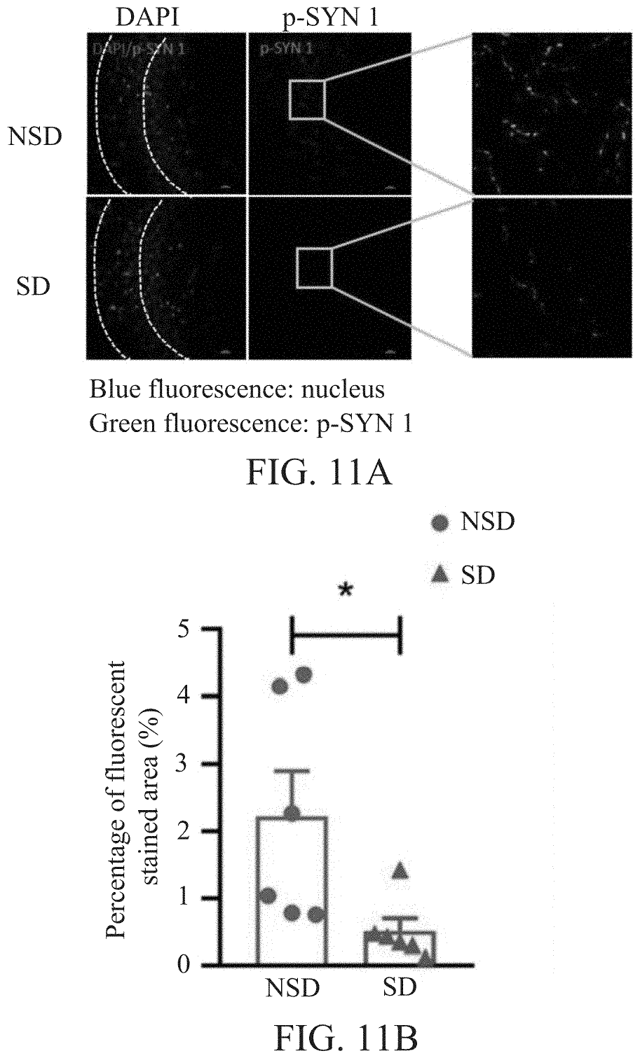

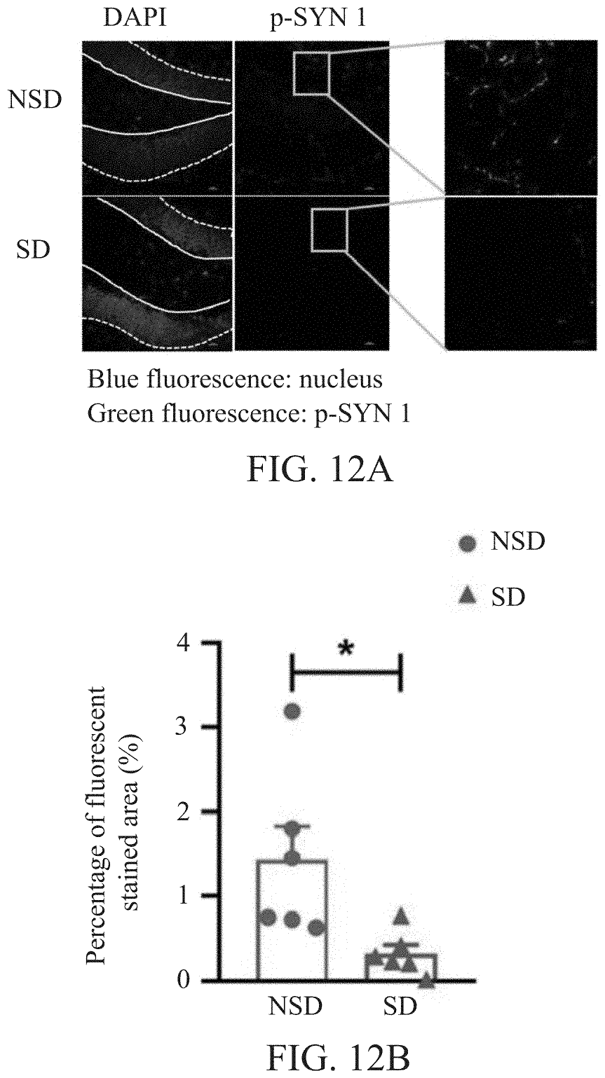

FIG. 9A, FIG. 9B, FIG. 10A, FIG. 10B, FIG. 11A, FIG. 11B, FIG. 12A and FIG. 12B show the display in the immunofluorescence staining analysis, the content of phosphorylated SYN 1 in the SD group was significantly higher in CA1 (p=0.01) (NSD: n=5 slices/3 experimental mice; SD: n=6 slices/3 experimental mice) (FIG. 9A and FIG. 9B), CA2 (p=0.04) (NSD: n=4 slices/3 experimental mice; SD: n=5 slices/3 experimental mice) (FIG. 10A and FIG. 10B), CA3 (p=0.03) (NSD: n=6 slices/3 experimental mice; SD: n=6 slices/3 experimental mice) (FIG. 11A and FIG. 11B) of hippocampus, and dentate gyms (DG) (p=0.08) (NSD: n=6 slices/3 experimental mice; SD: n=6 slices/3 experimental mice) (FIG. 12A and FIG. 12B) were lower than those in the NSD group. The results demonstrate impaired presynaptic function of the hippocampus in the SD group.

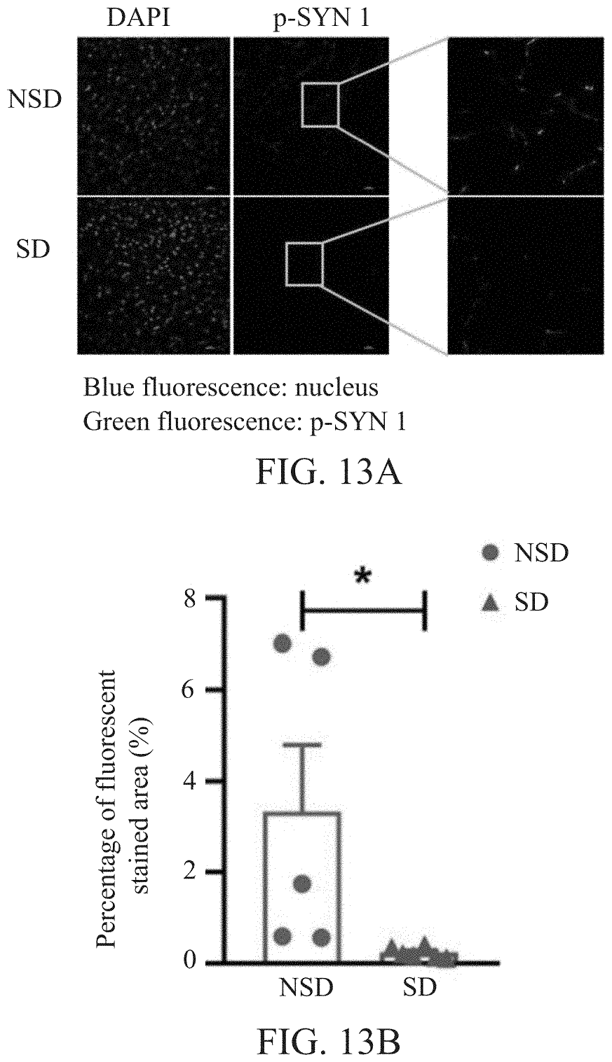

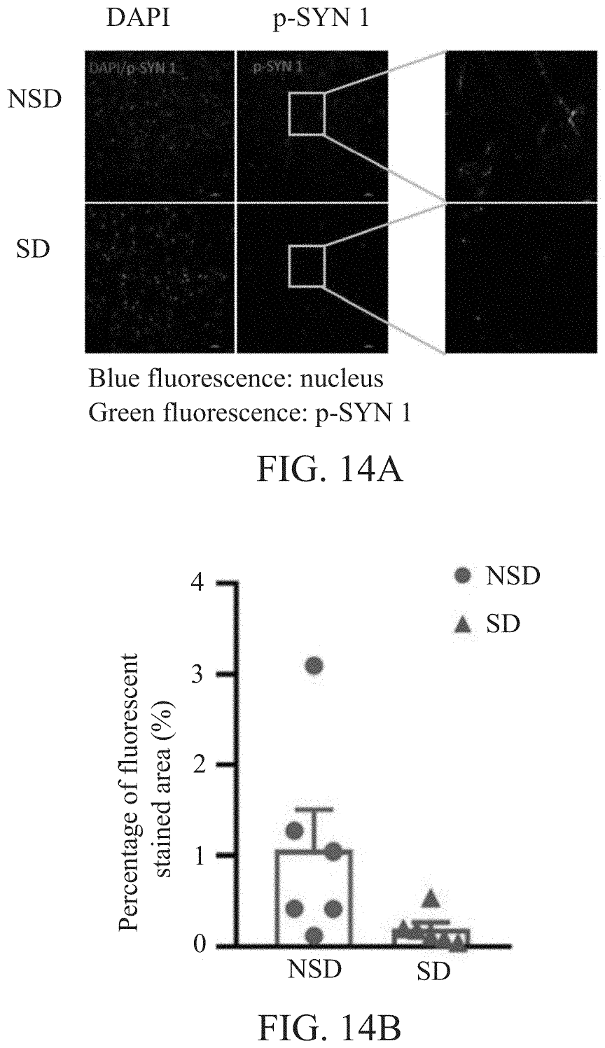

FIG. 13A, FIG. 13B, FIG. 14A and FIG. 14B show that in the immunofluorescence staining analysis, the content of phosphorylated SYN 1 (p-SYN 1) in the SD group was at Cortex (p=0.08) (NSD: n=5 slices/3 mice; SD: n=6 slices/3 mice) (FIG. 13A and FIG. 13B), and amygdala (p=0.04) (FIG. 14A and FIG. 14B) (NSD: n=6 slices/3 experimental mice; SD: n=6 slices/3 experimental mice) were significantly lower than those in the NSD group. The results demonstrate impaired presynaptic function in the amygdala and cortex of the experimental mice in the SD group.

6. Determination of Whether the Content of Gelsolin (GSN) and Related Proteins Changes Before Memory Retrieval:

After confirming from the molecular level that sleep deprivation can cause impaired presynaptic transmission in the hippocampus, amygdala, and cortex of the brain, it was determined whether the content of gelsolin and related proteins changed before Ret-1.

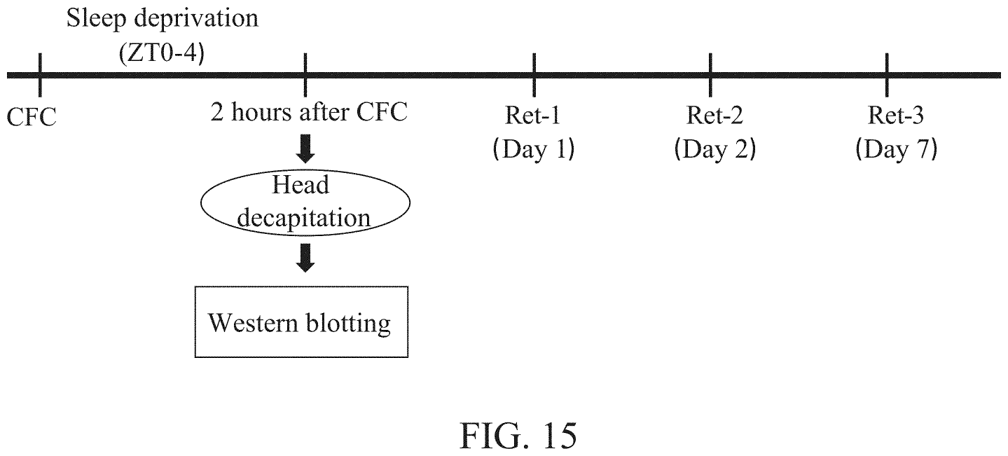

To determine whether the content of gelsolin (GSN) and related proteins changes before memory retrieval, hippocampal samples of the experimental mice were collected 2 hours after contextual fear training before Ret-1. The content of GSN, upstream targets of GSN, and the content of synapse-related proteins were confirmed by Western blot analysis. Among them, synapse-related proteins include PSD-95 and m-BDNF. The experimental procedure was shown in FIG. 15.

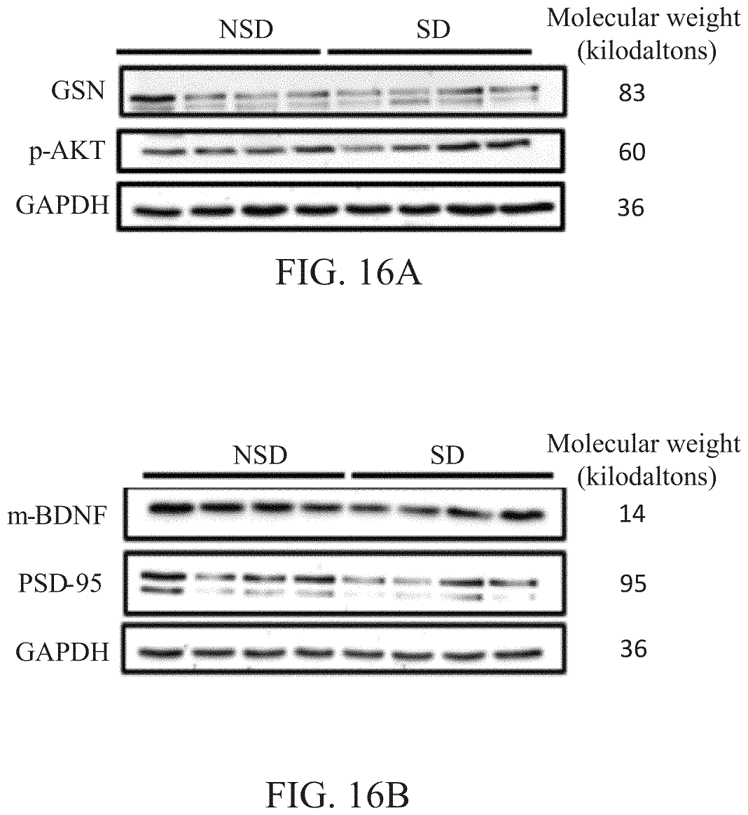

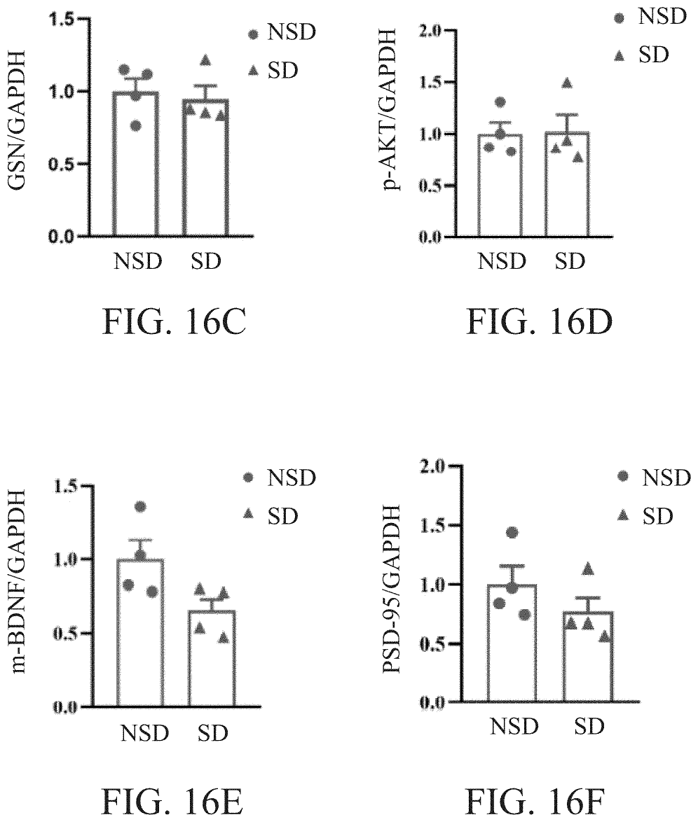

FIG. 16A, FIG. 16B, FIG. 16C, FIG. 16D, FIG. 16E and FIG. 16F are shown in Western blot analysis (NSD: n=5; SD: n=5), before memory retrieval performing on 2 hours after training, the content of GSN (p=0.695) (FIG. 16C), the content of phosphorylated AKT (p-AKT) (p=0.919) (FIG. 16D), the content of mature BDNF (m-BDNF) (p=0.06) (FIG. 16E), and the content of PSD-95 (p=0.281) (FIG. 16F), the hippocampus of the experimental mice in SD group and NSD group, after correction by GAPDH content, was not significantly different. The results demonstrate that sleep deprivation does not affect the structural molecular representation of synapses prior to memory retrieval.

7. Determination of Whether the Content of Gelsolin and Related Proteins Changes After Remote Fear Memory Retrieval:

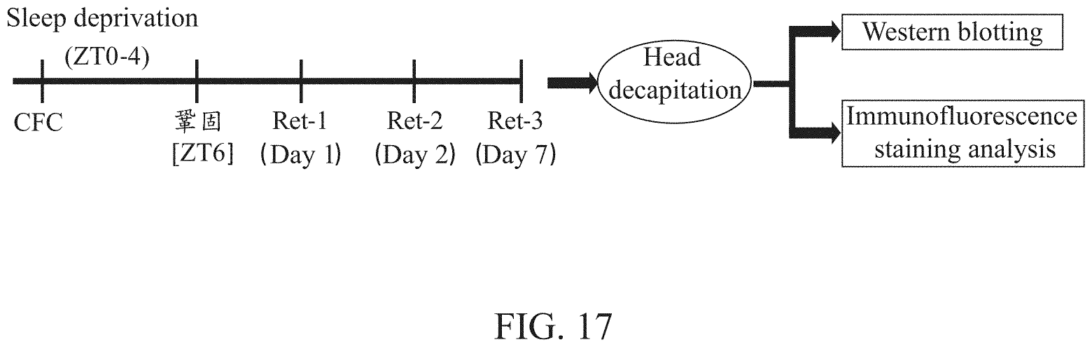

After determining that sleep deprivation does not affect the expression of synapse-related structural molecules before memory retrieval, it was next to determine whether the contents of GSN and related proteins changed after remote fear memory retrieval. Whole brain samples were collected after Ret-3, and the contents of GSN, upstream targets of GSN, and synapse-related proteins were confirmed by Western blot analysis. The upstream target of GSN is p-AKT. The synapse-related proteins include PSD-95 and m-BDNF. The schematic diagram of the experimental procedure is shown in FIG. 17.

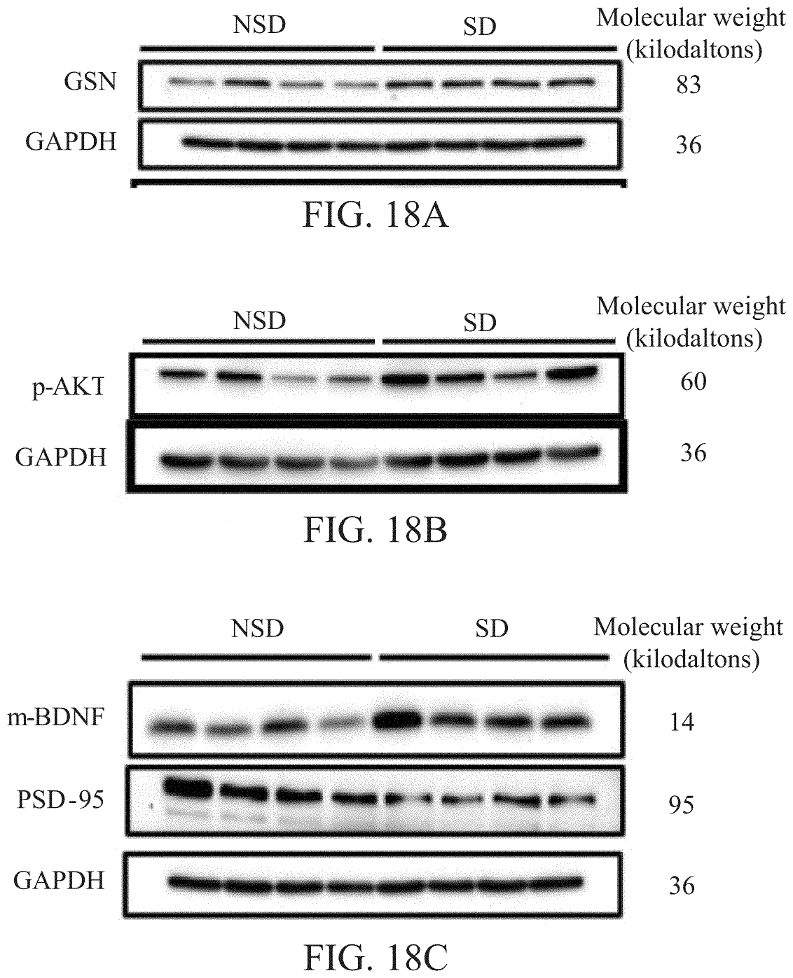

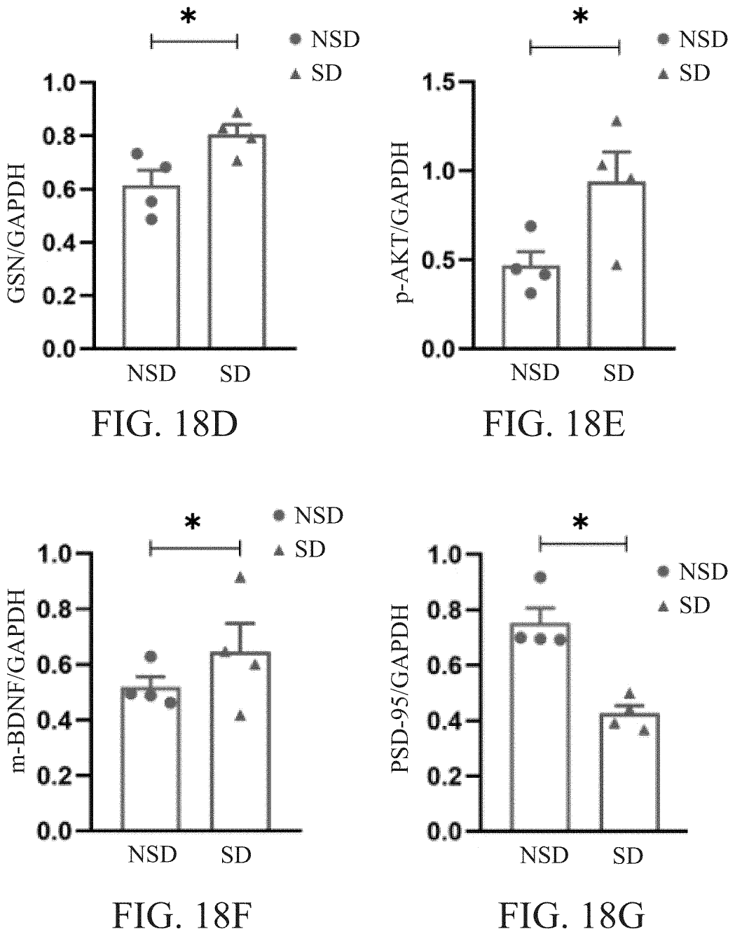

FIG. 18A, FIG. 18B, FIG. 18C, FIG. 18D, FIG. 18E, FIG. 18F and FIG. 18G are shown the data of each group after correction by the content of GAPDH. In Western blot analysis (NSD: n=5; SD: n=5), after Ret-3, the content of GSN (p=0.023) (FIG. 18D), the content of p-AKT (p=0.013) (FIG. 18E), and the content of m-BDNF (p=0.023) (FIG. 18F) in the experimental mice in the SD group was higher than those in the NSD group, all increased significantly. While the content of PSD-95 (p=0.019) (FIG. 18G) was significantly decreased, which demonstrate the impaired postsynaptic function of experimental mice in the SD group.

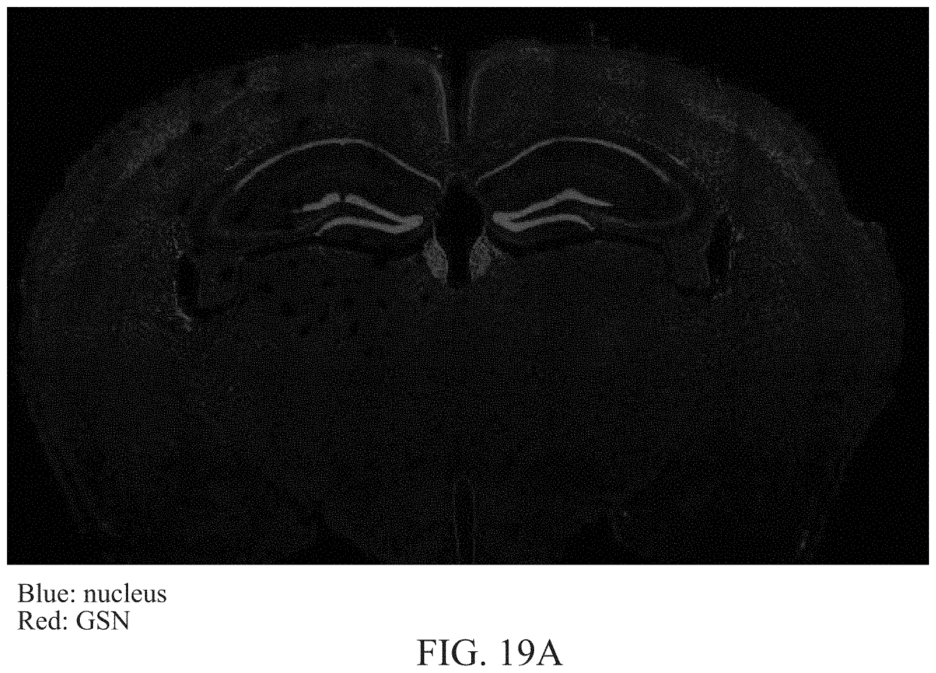

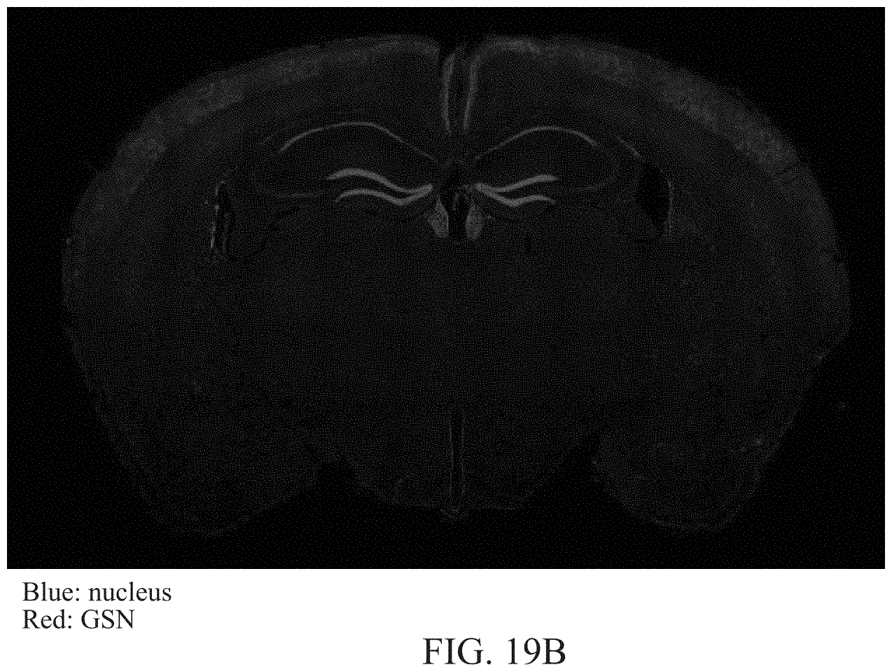

FIG. 19A and FIG. 19B respectively show the fluorescence staining diagrams of GSN in whole brain slices after the remote fear memory retrieval test in the experimental mice of the NSD group and the experimental mice of the SD group. It is shown that compared with the NSD group, the GSN in the SD group increased in different brain regions, including: cortex, superior thalamic habenula, hippocampus, thalamus, amygdala, and caudoputamen. The results demonstrate that sleep deprivation affects the synaptic-related structural molecular representation of long-range fear memory retrieval.

8. Determination of the Location of Gelsolin in the Brain After the Remote Fear Memory Retrieval Test:

After determining that sleep deprivation affects the synapse-related structural and molecular performance of remote fear memory retrieval, the location of GSN distribution in the brain after the remote fear memory retrieval test was further determined. Hippocampal samples were collected after Ret-3 for immunofluorescence staining analysis. All images were taken at 20× and 40× magnifications.

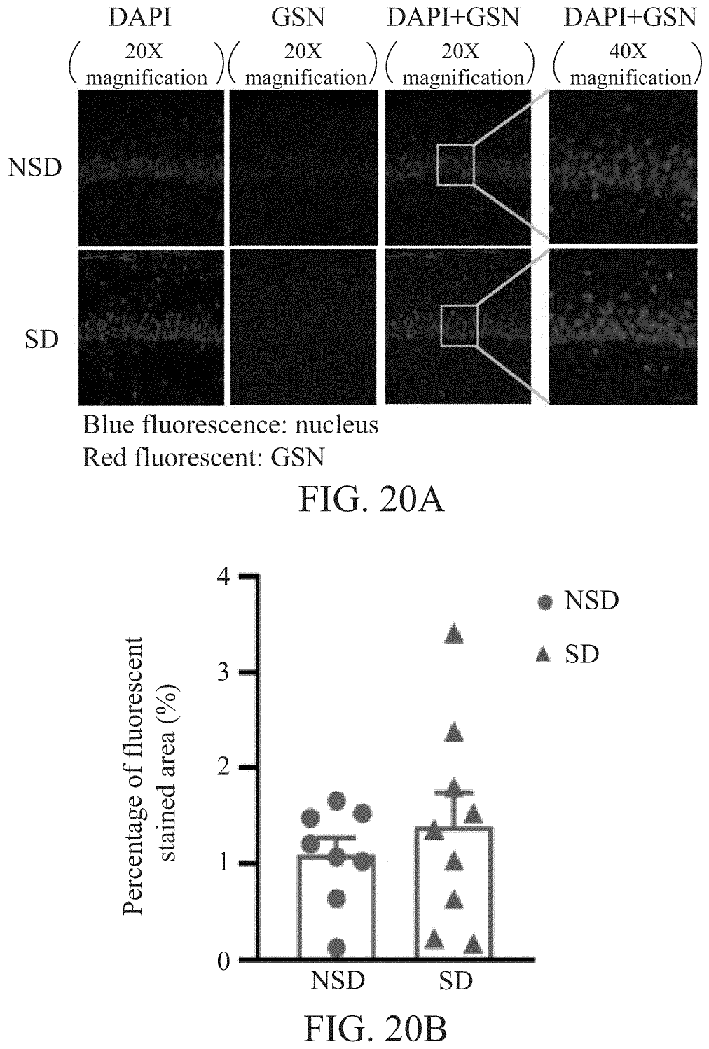

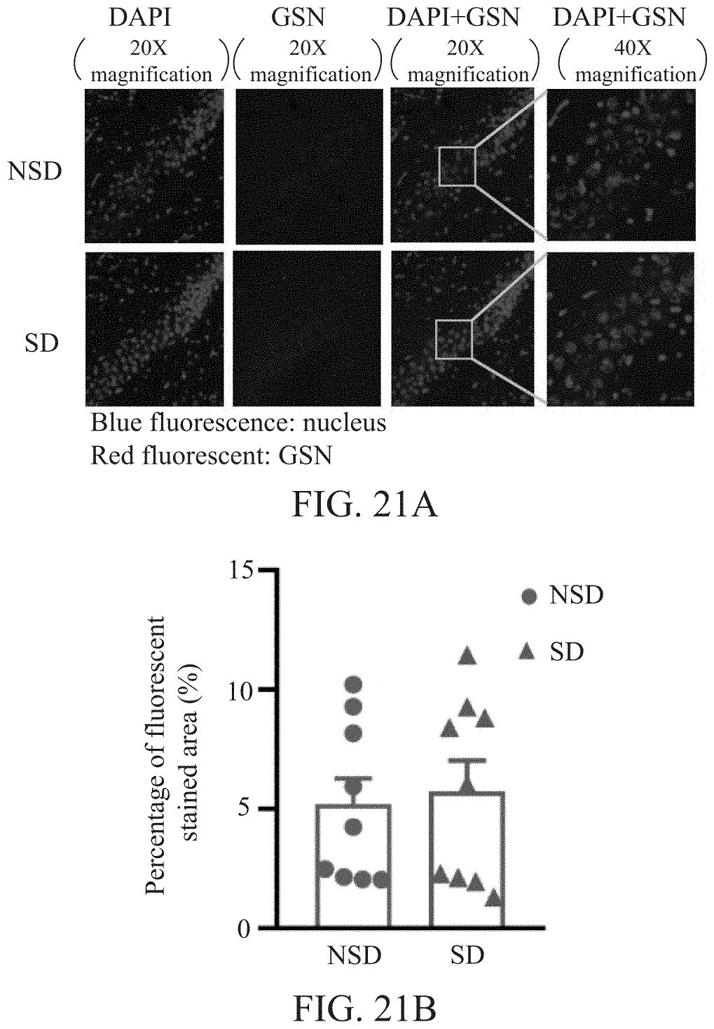

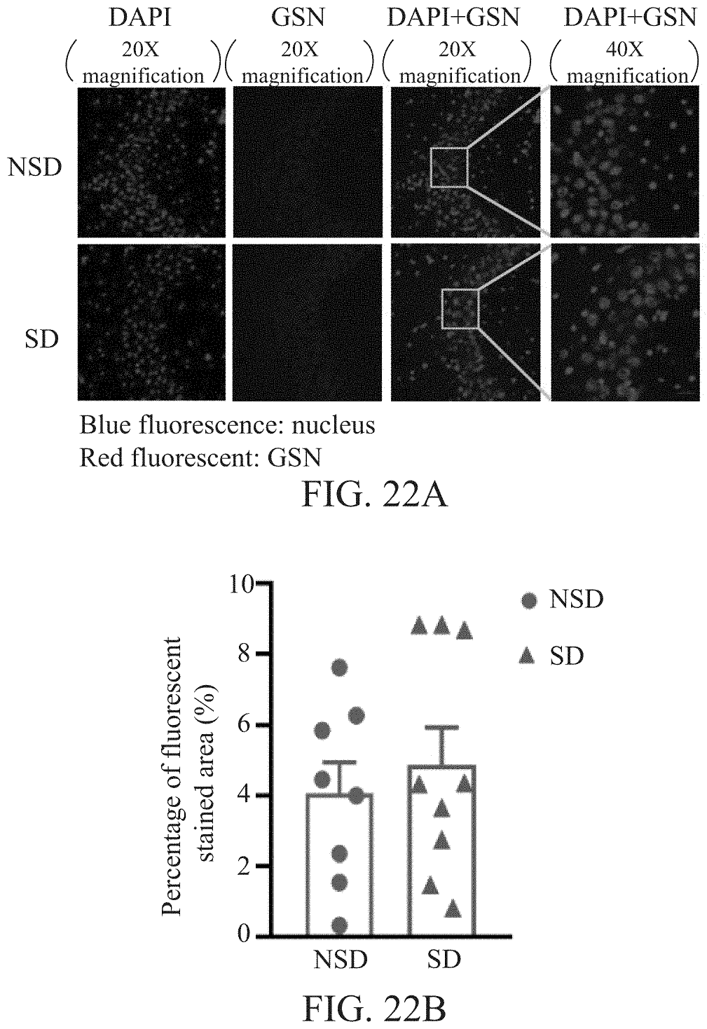

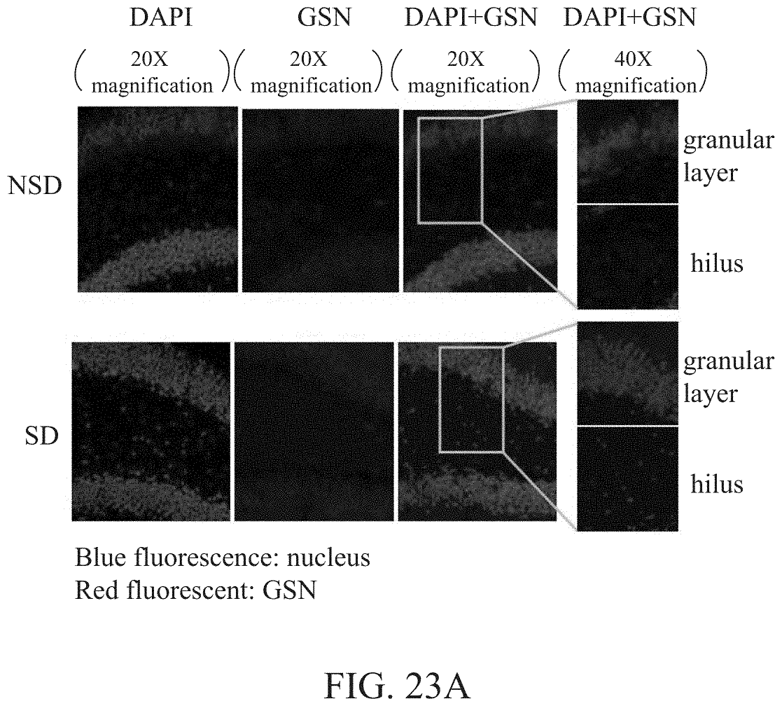

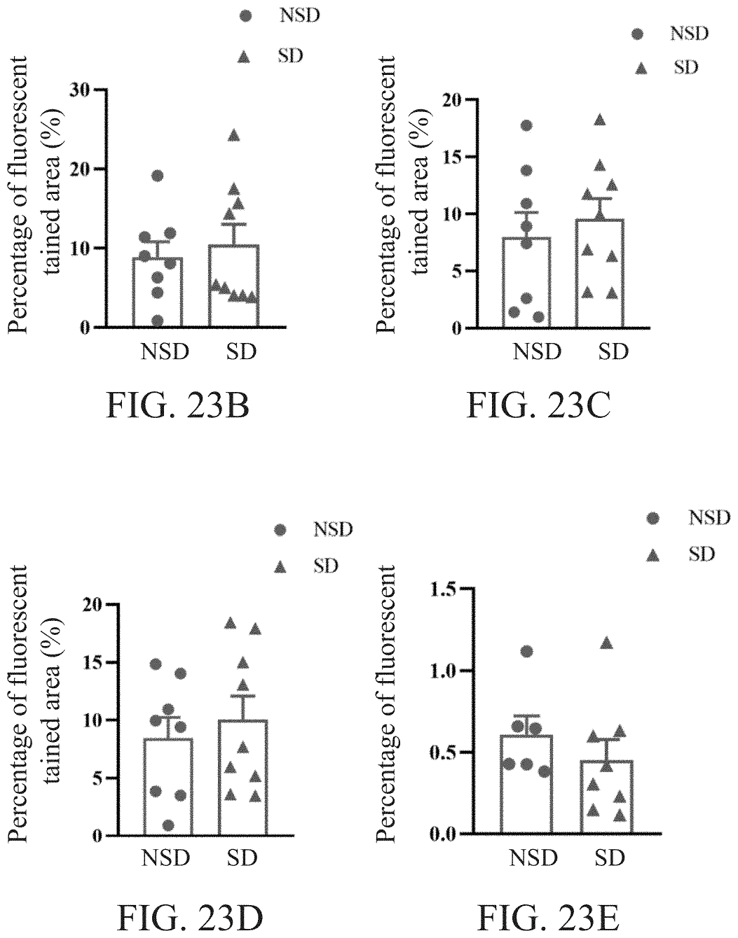

FIG. 20A, FIG. 20B, FIG. 21A, FIG. 21B, FIG. 22A, FIG. 22B, FIG. 23A, FIG. 23B, FIG. 23C, FIG. 23D and FIG. 23E show the content of GSN in SD group compared with the content of GSN in NSD group (NSD: 9 slices/3 experimental mice; SD: 9 slices/3 experimental mice), CA1 (p=1.09) (FIG. 20A and FIG. 20B), CA2 (p=0.37) (FIG. 21A and FIG. 21B), CA3 (p=0.28) (FIG. 22A and FIG. 22B) of hippocampus as well as the superior granular layer (p=0.27) (FIG. 23A) and the inferior granular layer Inferior granular layer (p=0.31) (FIG. 23B) and overall granular layer (FIG. 23C) of the dentate gyrus of the hippocampus showed an upward trend, while the hilus of the hippocampus (FIG. 23D) showed an downward trend (FIG. 23E).

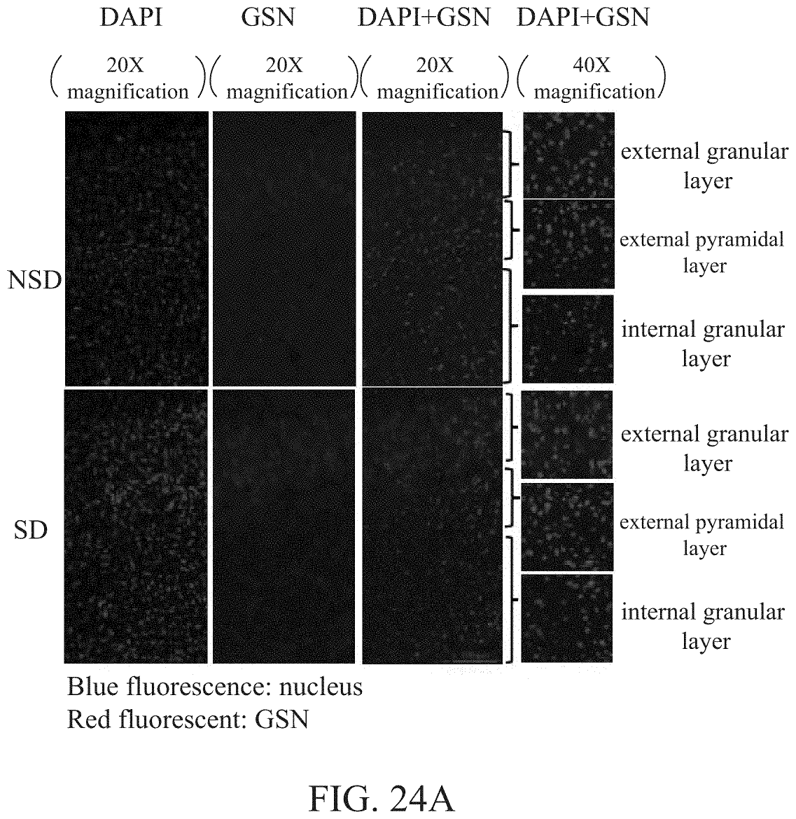

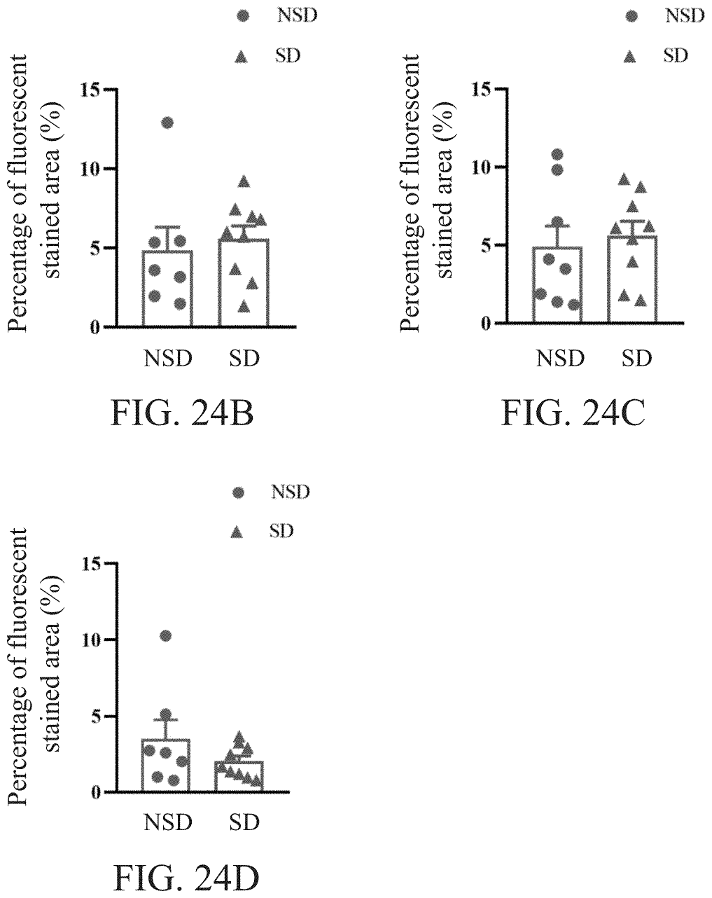

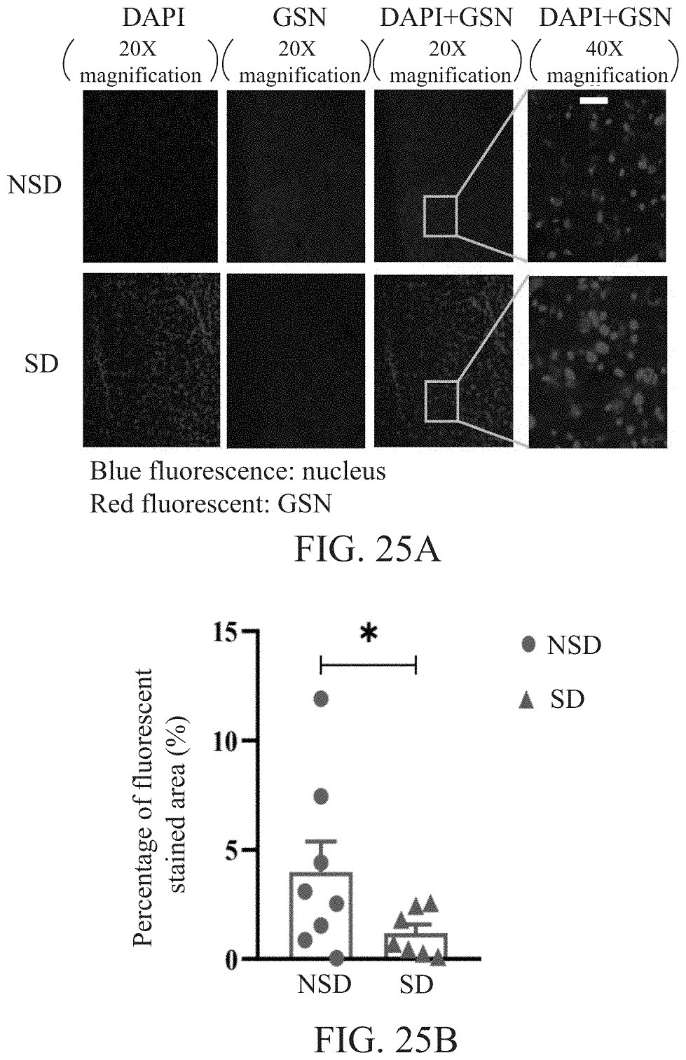

FIG. 24A, FIG. 24B, FIG. 24C, FIG. 24D, FIG. 25A and FIG. 25B show the content of GSN in SD group compared with the content of GSN in NSD group (NSD: 9 slices/3 experimental mice; SD: 9 slices/3 experimental mice), in the external granular layer (p=0.329) (FIG. 24B) and the external pyramidal layer (p=0.328) (FIG. 24C) of the brain cortex both showed an upward trend, while the internal granular layer (p=0.11) of the cortex (FIG. 24D) as well as amygdala (p=0.04) (FIG. 25A and FIG. 25B) showed a downward trend. The results demonstrate that in the experimental mice in the SD group, the content of GSN in most cortical regions of the brain showed an increasing trend.

9. Determination of Whether Sleep Deprivation Causes Actin Depolymerization After Remote Fear Memory Retrieval Test:

After determining the location of gelsolin in the brain after the remote fear memory retrieval test, it was further determined whether sleep deprivation caused actin depolymerization after the remote fear memory retrieval test.

To determine whether sleep deprivation causes actin depolymerization after the remote fear memory retrieval test, immunohistochemical analysis was used to determine the content of filamentous actin (F-actin) in experimental mice in the group in SD group and NSD group after the remote fear memory retrieval test was performed. All images were taken at 10× and 40× magnifications.

Immunohistochemical Analysis:

In the immunohistochemical analysis, first, the brain slices were immersed in 0.1% paraformaldehyde fix solution (PFA) for preservation. Then, the slices were rinsed with PBS for 5 minutes and rinsed with a non-xylene solution (Humuto Chemical Co., Ltd) for 5 minutes. Next, the non-xylene solution was removed and the slices were dehydrated in 85% ethanol for 30 seconds. Next, 85% ethanol was removed and the slices were rinsed with PBS for 10 minutes. Next, the tissue was immersed in citrate buffer at 95° C. for 30 minutes. Next, the citrate buffer was removed and the sections were immersed in a hydrogen peroxide block for 10 minutes at room temperature. Next, the hydrogen peroxide blocking solution was removed and the slices were rinsed 3 times with PBS for 10 minutes each. Next, the slices were immersed in high-efficiency blocking agent (Ultra V block, Thermo Fisher Scientific, USA) for 5 minutes, and then rinsed with PBS 3 times for 10 minutes each time. Next, the slices were treated with a primary antibody (1:100) (LSBio, USA) recognizing filamentous actin (F-actin) (LSBio, USA) at 4° C. for 18 hours, and rinsed with PBS 3 times for 10 minutes each time after removing the primary antibody dilution. Next, the slices were immersed in primary antibody amplifier Quanto (Thermo Fisher Scientific, USA) for 10 minutes at room temperature, and rinsed with PBS 3 times for 10 minutes each time. Next, the slices were immersed in horseradish peroxidase reagent (HRP polymer Quanto, Thermo Fisher Scientific, USA) for 10 minutes at room temperature in the dark, and then rinsed with PBS 3 times for 10 minutes each time. Next, the slices were immersed in Diaminobenzidine (DAB) for 20 seconds. Finally, the slices were attached to a slide and covered with a cover slip for observation. Slice imaging was observed using bright field microscopy. Data quantification was analyzed with image processing software (Image J software) and graphed with graphing software (GraphPad Prism 8). Image cropping and contrast adjustment were performed using image processing software (Adobe photoshop).

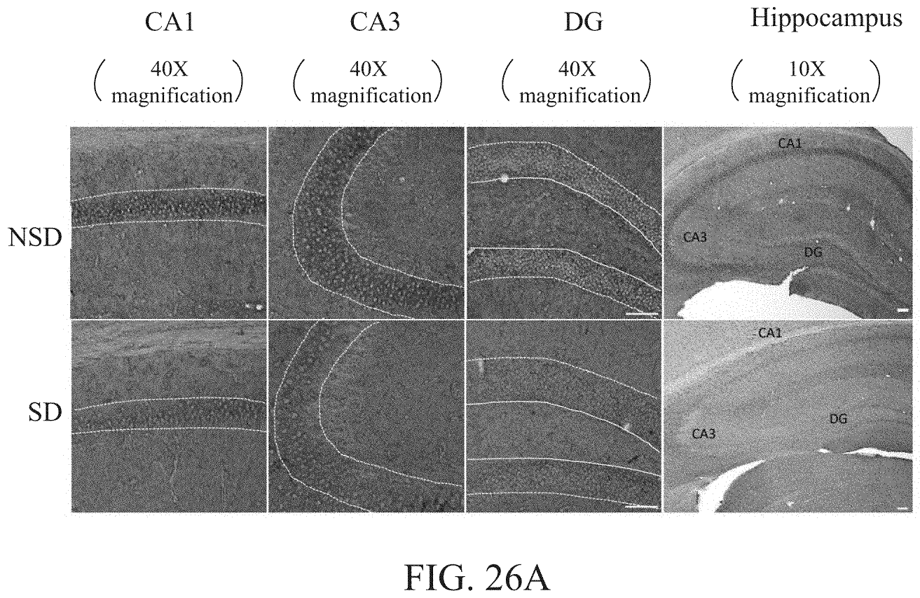

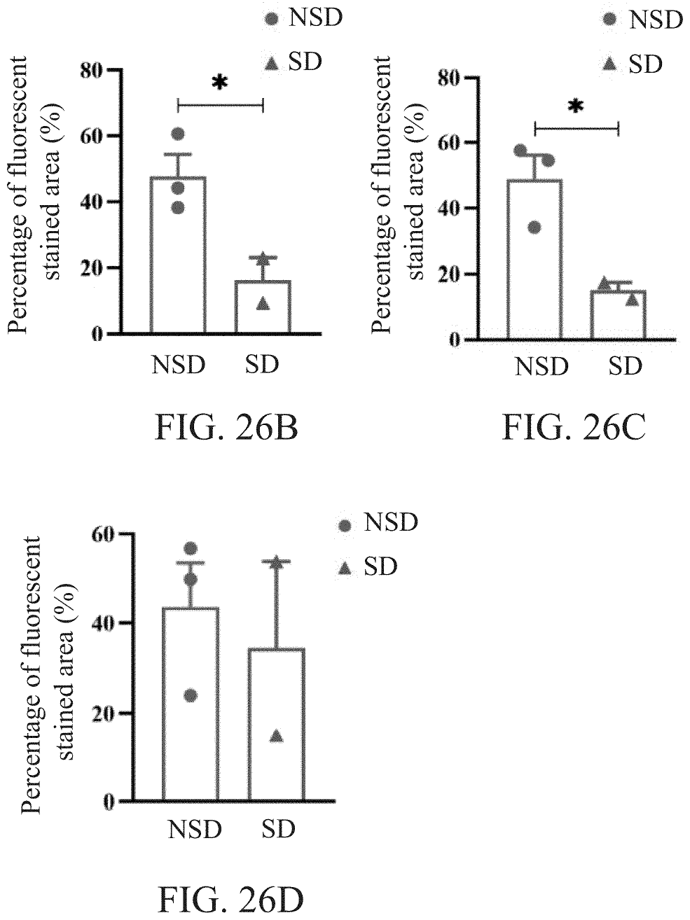

FIG. 26A, FIG. 26B, FIG. 26C, FIG. 26D show the content of F-actin in CA1 (p=0.05) (FIG. 26B), CA3 (p=0.04) (FIG. 26C) and DG (p=0.06) (FIG 26D) of the hippocampus showed an downward trend, especially in CA1 and CA3 of the hippocampus. The results demonstrate that in the experimental mice in the SD group, actin depolymerization was increased, and this result was positively correlated with the aforementioned increase in GSN.

10. Determination of Whether Reducing the Content of GSN can Improve the Problem of Memory Degradation Caused by Sleep Deprivation:

After determining whether sleep deprivation causes actin depolymerization after the remote fear memory retrieval test, it was determined whether reducing the content of GSN can improve the problem of memory degradation caused by sleep deprivation.

Stereotaxic Infusion:

First, the mice were injected with an anesthetic drug via intravenous injection. Among them, the anesthetic drug consisted of 0.64 mL of ketamine, 0.4 mL of xylazine, and 9.36 mL of 0.9% saline. After 20 minutes of anesthesia, first, the hair above the skull of the experimental mice was removed to expose the scalp, and tetracycline HCl was applied to the eyes to prevent drying. Next, the mice were fixed in a stereotaxic apparatus, a 1-inch incision was made above the skull, and iodine was used to prevent infection. Anterior-posterior (AP), medial-lateral (ML), and dorsal-ventral (DV) coordinates were performed using a guide cannula for record of the bregma. Three plane coordinates were determined according to the mouse-brain atlas. According to the coordinates, two positions (AP=−1.5 mm, ML=+/−1.5 mm) in the brain of the experimental mice were drilled with 0.1 mm diameter holes, and the positions were recorded using a catheter. The catheter was replaced with an injection cannula and connected to a 100 μL syringe (syringe) fixed to a syringe pump. The syringe was placed at the above coordinates and at the depth of the hippocampal location (DV=−0.8 mm). After completing the setup, in order to test the accuracy of site injection, Coomassie blue dye was injected into the hippocampus on both sides of the brain using a syringe pump at a flow rate of 1.5 μL/min, and the opening was sutured. The experimental mice were immediately decapitated, and brain slices were performed to confirm the location of the dye. Finally, the final coordinates (AP=−1.5 mm, LM=+/−1.5 mm, and DV=−0.8 mm below bregma) for subsequent injections of the inhibitor of actin recombination modulator were determined by adjustment tests.

Preparation of Inhibitors of Actin Recombination Regulators:

In the preparation of the inhibitor of actin recombination regulator, the purchased GSN siRNA (s105802, Thermofisher Ambion, Life technologies cooperation, USA) with an original concentration of 5 nanomolar (nmole) was prepared in nuclease-free water (Nuclease-free water) to dilute the original concentration to the working concentration, i.e. 1 μg/μL of GSN siRNA, and then 1 μg of GSN siRNA was injected into the hippocampus on both sides of the brain of experimental mice. Among them, the molecular weight of GSN siRNA is 13,400 Daltons (Da). Among them, GSN siRNA was used to inhibit the expression of GSN gene (chromosome 2: 35256359-35307902 on Build GRCm38) in experimental mice, and reduce the expression of GSN protein. After the opening was sutured, the mice were injected with 1 mL of 0.9% normal saline and painkiller (meloxicam), and then the experimental mice were placed back into the cages and the conditions of the experimental mice were monitored for 2 days.

In order to determine whether reducing the content of GSN can improve the problem of memory degradation caused by sleep deprivation, GSN siRNA was directly injected into the hippocampus of the experimental mice in the SD group, thereby reducing the content of GSN by siRNA. Another group of the experimental mice in the SD group was injected with scrambled siRNA as a negative control group (SD+Scramble: n=3; SD+siRNA: n=4), and the content of GSN in hippocampus and amygdala on the 7th and 13th days were observed. In the following figures, the negative control group is denoted by “NC”, and the group injected with GSN siRNA is denoted by “GSN siRNA”.

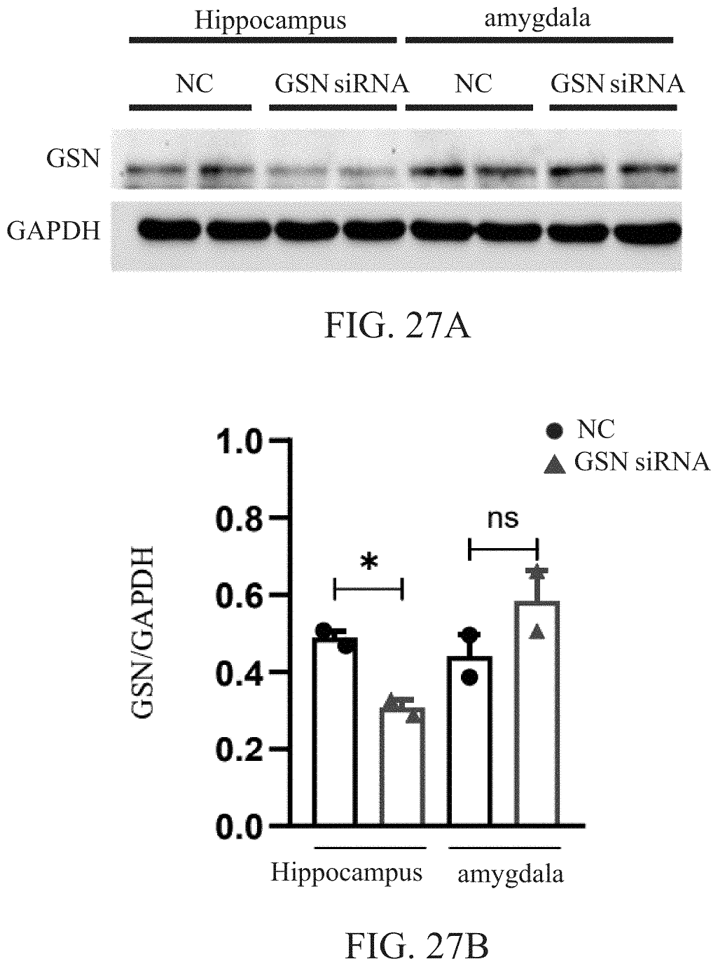

FIG. 27A and FIG. 27B show that GSN siRNA can significantly inhibit the content of GSN in the hippocampus on the 7th day but not the content of gelsolin (p=0.27) in the amygdala, after correction by the content of GAPDH (p=0.023); “ns” in FIG. 27A and FIG. 27B demonstrates a statistically significant difference respectively.

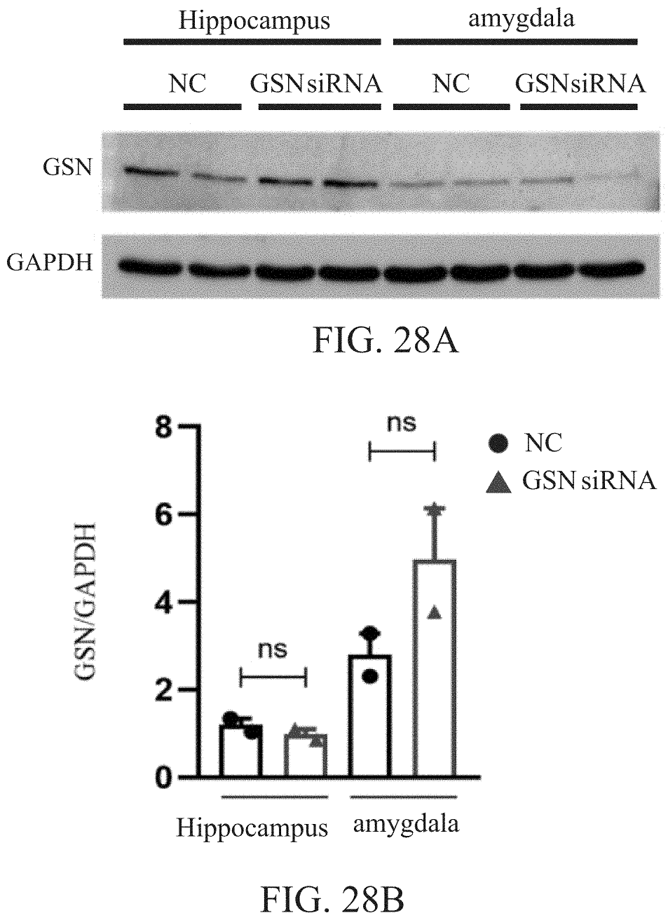

FIG. 28A and FIG. 28B show that on the 13th day, GSN siRNA did not significantly change the content of GSN in the hippocampus and amygdala after correction by the content of GAPDH; “ns” in FIGS. 28A and 28B demonstrates a statistically significant difference respectively.

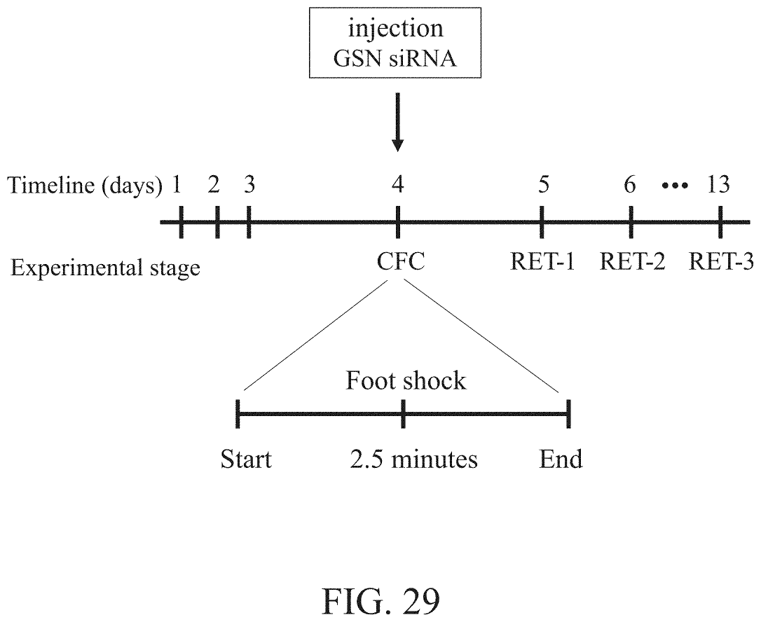

After determining that GSN siRNA injection could reduce the content of GSN in the hippocampus of experimental mice on the 7th day, before the contextual fear conditioning experiment, GSN siRNA was injected into the experimental mice in the SD group and recovered after two days of rest. Then, the contextual fear conditioning experiment was performed, and the performance of retrieval, reconsolidation, and remote fear memory retrieval was compared with those of the experimental mice in the SD group without GSN siRNA injection. The experimental procedure is shown in FIG. 29.

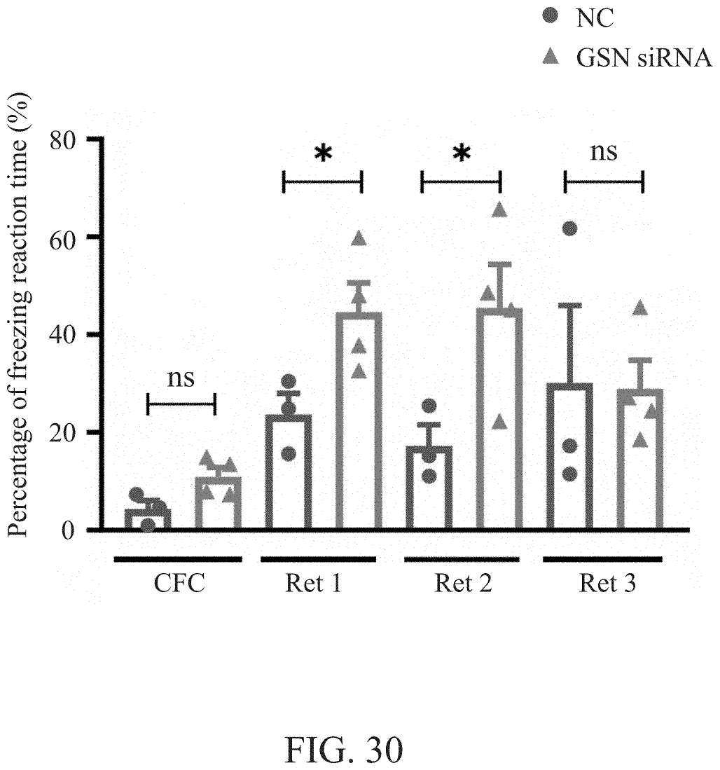

FIG. 30 shows that injection of GSN siRNA reversed the fear memory degradation induced by SD in Ret-1 (p=0.04) (retrieval) and Ret-2 (p=0.05) (reconsolidation), but not in Ret-3 (remote fear memory retrieval) was not significantly different from the control group; “ns” in FIG. 30 indicates that a statistically significant difference was not reached.

Through the above examples, it can be determined that the memory deterioration caused by sleep deprivation can be improved by inhibiting the content of gelsolin (actin recombination regulator).

The above is only to provide a preferred embodiment to disclose the content of the present disclosure, but it is not intended to limit the present disclosure. Any modifications that can be easily thought of by those with ordinary knowledge in the technical field to which the present disclosure pertains also fall into the inventive concept and the claim scope of the patent application.

What is claimed is:

1. A use of an inhibitor of actin remodeling modulator for the preparation of a medicament for treating memory deterioration caused by sleep deprivation.

2. The use of the inhibitor of actin recombination regulator according to claim 1, wherein the actin recombination regulator is gelsolin.

3. The use of the inhibitor of actin recombination regulator according to claim 2, wherein the inhibitor of actin recombination regulator comprises shRNA, miRNA, siRNA, antibody, antagonist or combination thereof.

4. The use of the inhibitor of actin recombination regulator according to claim 3, wherein the inhibitor of actin recombination regulator is siRNA.

5. The use of the inhibitor of actin recombination regulator according to claim 2, wherein the mode of administration of the inhibitor of actin recombination regulator is selected from the group consisting of: intracerebroventricular administration, intracerebral administration, intrathecal administration, arterial administration, intradermal administration, intramuscular administration, intragastric administration, intraperitoneal administration, intravenous administration, oral administration, subcutaneous administration, topical administration, systemic administration.

6. The use of the inhibitor of actin recombination regulator according to claim 5, wherein the mode of administration of the inhibitor of actin recombination regulator is selected from the group consisting of: intracerebroventricular administration, intracerebral administration, intrathecal administration.

7. The use of the inhibitor of actin recombination regulator according to claim 2, wherein the inhibitor of actin recombination regulator is further used in combination with hypnotic drug.

8. The use of the inhibitor of actin recombination regulator according to claim 7, wherein the hypnotic drug is selected from the group consisting of benzodiazepines, non-benzodiazepines, barbituric acid Salts, and the group consisting of melatonin receptor agonists.
